(19) United States
(12) Patent Application Publication
(10) Pub. No.: US 2006/0104901 A1
(43) Pub. Date: May 18, 2006

(54) Preventing or treating epithelial tissue damage or hair loss

(76) Inventors: Angus Moodycliffe, Pully (CH); Laure Poquet, Sur-Lausanne (CH)

Correspondence Address: BELL, BOYD & LLOYD LLC, P. O. BOX 1135, CHICAGO, IL, 60690-1135, US

(21) Appl. No.: 10/525,256

(22) PCT Filed: Aug. 27, 2003
(86) PCT No.: PCT/EP03/09496

§ 371 Date: Sep. 13, 2005

(30) Foreign Application Priority Data
Aug. 27, 2002 (EP) 020192449

(51) Int. Cl. A61K 49/00; A61K 48/00; A61K 31/737; A61K 31/715; A61K 8/64

(52) U.S. Cl. 424/9.1; 514/44; 514/54; 424/439; 424/70.14

(57) ABSTRACT

The present invention pertains to a method for preventing and/or treating epithelial tissue damage, such as is effected by inflammatory reactions, ageing or cancer and/or to prevent and/or treat hair loss. In particular, the present invention relates to substances and/or compositions modifying, in particular blocking endogenous CD_{1d} function. According to another aspect the present invention also provides a method for screening for compounds suitable for use in the method and the composition of the present invention.

### BACKGROUND OF THE INVENTION

The present invention pertains to a method for preventing and/or treating epithelial tissue damage, such as is effected by inflammatory reactions, ageing or cancer and/or to prevent and/or treat hair loss. In particular, the present invention relates to substances and/or compositions modifying, in particular blocking endogenous CD_{1d} function. According to another aspect the present invention also provides a method for screening for compounds suitable for use in the method and the composition of the present invention.

### SUMMARY OF THE INVENTION

The most prominent epithelial tissue in living beings is the skin, which represents the largest organ in the organism. The system of skin integument, which comprises the epidermis, dermis and the stratum cornium, correlates with those of internal organs and concurrently interacts with the surroundings. Being the interface between the environment and organism itself, the skin is heavily influenced by external factors and also variable parameters of the organism's inner system. The skin's regulative mechanisms need, therefore, always be active to induce systemic changes necessary to maintain normal pathological events concerning skin integument morphology and activities. A great deal of processes assuring the adequate consumption of increased affluence of energetic and plastic substances according to the skin's needs become guarantors of morphological and functional stability of skin structures. So, the state of integuments determines the realization of metabolic processes necessary for skin cell viability and activity leading to the presence of healthy skin peculiarities such as barrier function, elasticity, turgor properties, humidity, pigmentation etc.

During the lifetime of a living being different signs, characteristic of ageing, appear on the skin, with the principal clinical signs being the appearance of fine lines and deep wrinkles which increase or are accentuated with age. Moreover, the skin's complexion is generally modified and diffuse irritations and occasionally telangiectasias may come into existence on certain areas.

These signs of ageing are even promoted by exposure of the skin to exogenous influences, such as e.g. UV-radiation, pollutants, free radicals or chemical substances.

Moderate UV exposure generally causes the well known effects of reddening the skin with an accompanying inflammation reaction, known as erythema. This phenomenon, often referred to as “sunburn”, is painful and commonly results in a subsequent peeling of the skin.

Moreover, excessive UV-exposure of the skin may also lead to the onset of severe disorders, such as carcinogenesis, the most common tumours being the basal cell carcinoma (BCC), followed by squamous cell carcinoma (SCC), and more rarely malignant melanoma. Apart from damages on the DNA-level also immuno-suppression caused by UV exposure seems to account for both, non-melanoma and melanoma cancer promotion. It is presently acknowledged that photo-induced immuno-suppression permits the initiated tumour cell to evade recognition and rejection by normal immunological mechanisms, to remain latent for extended periods, and to eventually proliferate into a tumour. This concept concurs with the findings that immuno-compromised patients, whether genetically (xeroderma pigmentosum) or pharmacologically, such as e.g. organ transplant recipients, have a higher incidence of skin cancer as compared to people with a properly functioning immune system.

In the art several means have been proposed to prevent destructive effects of environmental factors on epithelial cells, in particular skin epithelial cells.

As regards protection to sun radiation “sun blocks” or “sunscreens” have been made available, which are applied to the skin prior to sun exposure. Typically, sunscreen compositions contain chemical agents, such as certain benzophenones, dibenzylmethanes or substituted paraaminobenzoates, i.e. compounds absorbing ultraviolet radiation, so that it cannot penetrate the skin. However, some of the compounds used for this purpose have shown to lack sufficient light stability and may even become toxic over long term application. In addition, they must stay continuously on the surface of the skin at the time of exposure to be effective. However, sunscreens are easily rubbed off or washed off by sweating or swimming and can also be lost by penetration into the skin.

Another means to prevent skin deterioration or ageing, respectively, is to provide compounds scavenging free radicals. In this respect EP 0 761 214 discloses singlet oxygen quenchers comprising aniline derivatives and difurfuryl amine derivatives, which are reported to reduce the oxidative stress to the skin.

Yet, all these means and methods are not sufficiently capable to protect the skin from the growing challenge in our environment. To this contributes an increased atmospheric pollution and also social behaviour, according to which sun-tan is associated with health, beauty and status. As a consequence many people expose their skin to sun radiation to acquire a tan in spite of the negative results accompanying such behaviour being well known. This problem even gets more prominent with the ozone shield covering the earth becoming thinner, resulting in a heavier exposure of living beings to UV radiation.

Consequently there is a need in the art to provide a better protection of the skin to environmental factors, such as stress or sun radiation.

Accordingly, an object of the present invention is to obviate the drawbacks of the prior art and to provide such means in order to protect the skin from unfavourable influences encountered in the environment, in particular from oxidative or chemical stress or sun radiation.

This problem has been solved by providing a substance, that is capable to essentially modify, in particular block the endogenous CD_{1d} function in epithelial cells.

Additional features and advantages of the present invention are described in, and will be apparent from, the following Detailed Description of the Invention and the figures.

### BRIEF DESCRIPTION OF THE FIGURES

FIG. 1A. Wild-type mice exhibit skin damage (burning) following exposure to a single dose (86 mJ/m²) of UVB radiation.

FIG. 1B. Wild-type mice exhibit skin damage (burning) following exposure to a single dose (86 mJ/m²) of UVB radiation. (Close-up).

FIG. 1C. CD1d knockout mice show no obvious signs of skin damage following exposure to a single dose (86 mJ/m²) of UVB radiation.

FIG. 1D. CD1d knockout mice show no obvious signs of skin damage following exposure to a single dose (86 mJ/m²) of UVB radiation. (Close-up).

FIG. 2. Difference in degree of UVB-induced skin damage between wild-type (Right) and CD_{1d} knockout (Left) mice exposed to two doses (86 mJ/m²) of UVB radiation.

FIG. 2A. Damaged (lesions) dorsal skin of wild-type mice exposed to two doses (86 mJ/m²) of UVB radiation (Close-up).

FIG. 2B. Undamaged dorsal skin of CD_{1d} knockout mice exposed to two doses (86 mJ/m²) of UVB radiation.

FIG. 3. CD_{1d} knockout mice exhibit increased epidermal apoptosis in their dorsal epidermis compared to wild-type mice, as measured by TUNEL. Wild-type (A) and CD1d knockout (B) mouse skin not exposed to UV-irradiation. Wild-type (C) and CD_{1d} knockout (D) mouse skin 48 h after a single exposure (86 mJ/m²) to UV-B radiation.

FIG. 4 a and b are graphs indicating the approximate amount of CD_{1d} in different tissues in mice and human.

FIG. 5 shows that CD_{1d} protein is expressed in the epidermis of mouse skin 72 h following exposure to a single dose (430 mJ/cm²) of UVB radiation;

FIG. 6 shows that murine skin CD_{1d} gene transcription is regulated following UVB irradiation;

FIG. 7 shows that murine skin CD_{1d} gene transcription is regulated following solar simulated light irradiation;

FIG. 8 shows that CD_{1d} gene transcription in immortalized (DK7) human keratinocytes is regulated following solar UV irradiation;

FIG. 9. shows that COX-2 and TNF-alpha mRNA levels are down-regulated in UVB-irradiated CD_{1d} knockout mouse skin.

FIG. 10 a and b) show that mouse skin IL-6 and MIP1-alpha protein levels 48 h after UVB irradiation are significantly decreased in CD_{1d} KO mice.

FIG. 11 shows that hydrocortisone suppresses CD_{1d} transcription in cells exposed to a chemical stress; and

FIG. 12 shows that CD_{1d} is expressed in human hair follicles.

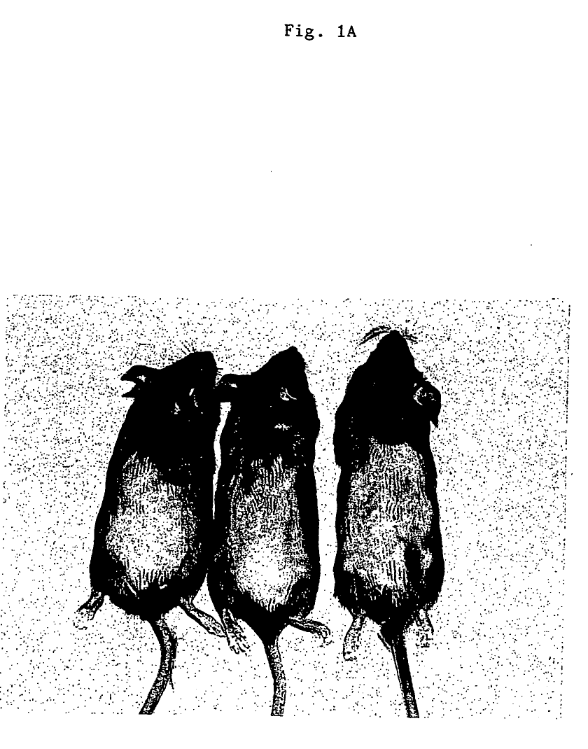

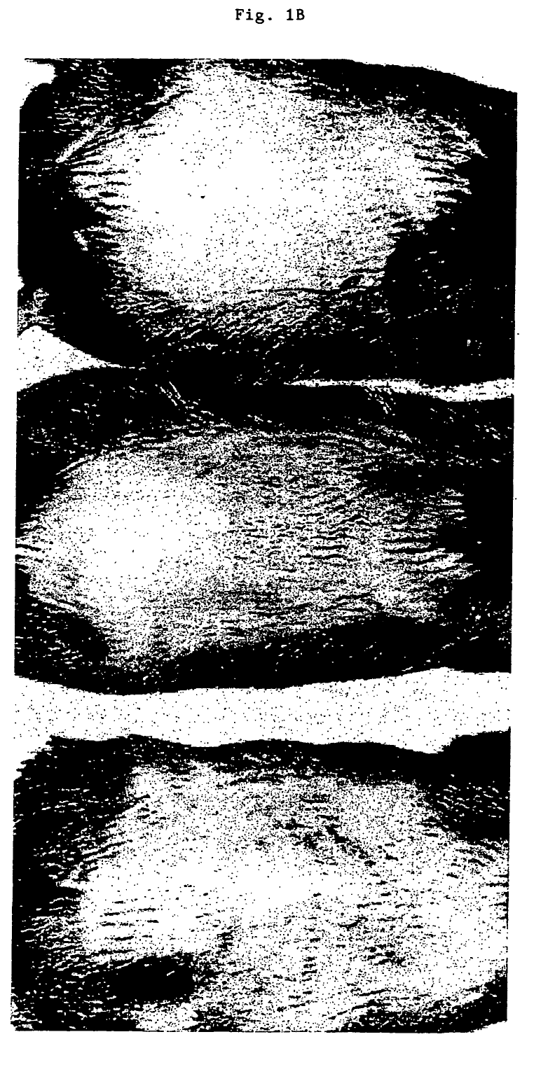

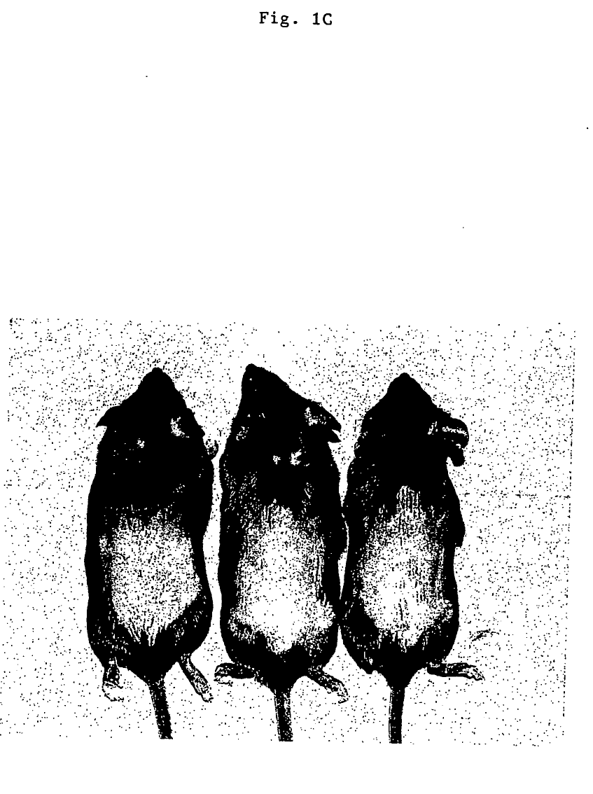

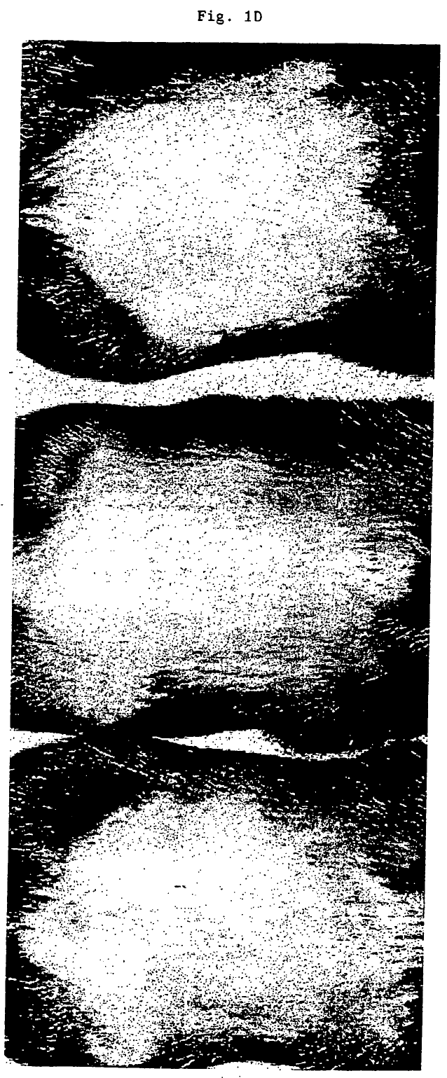

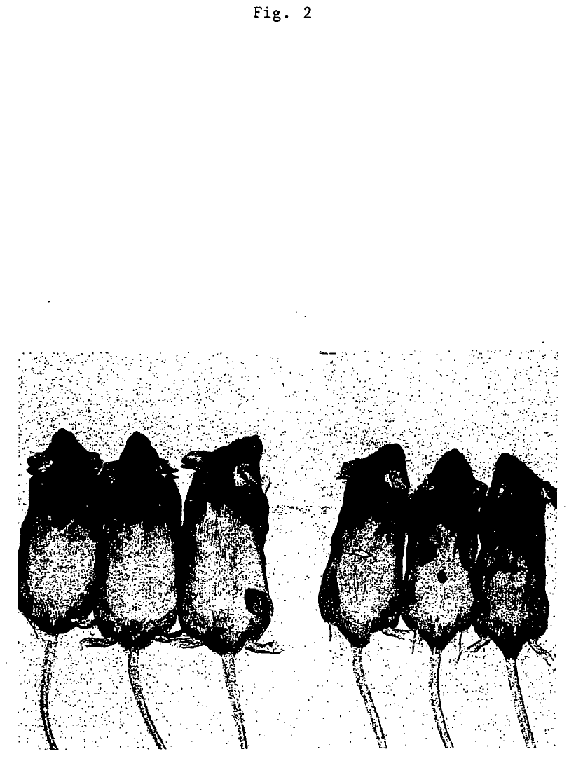

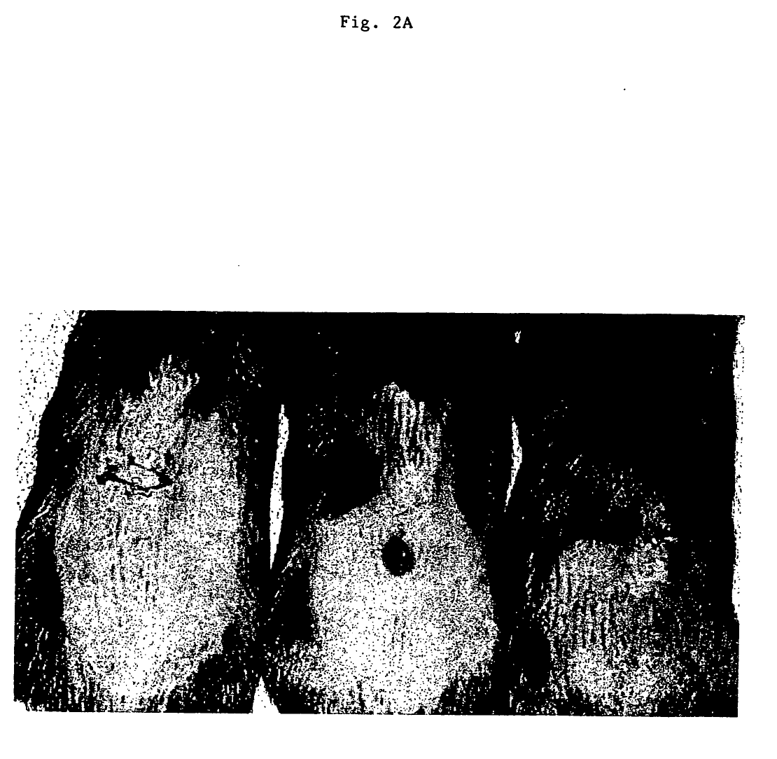

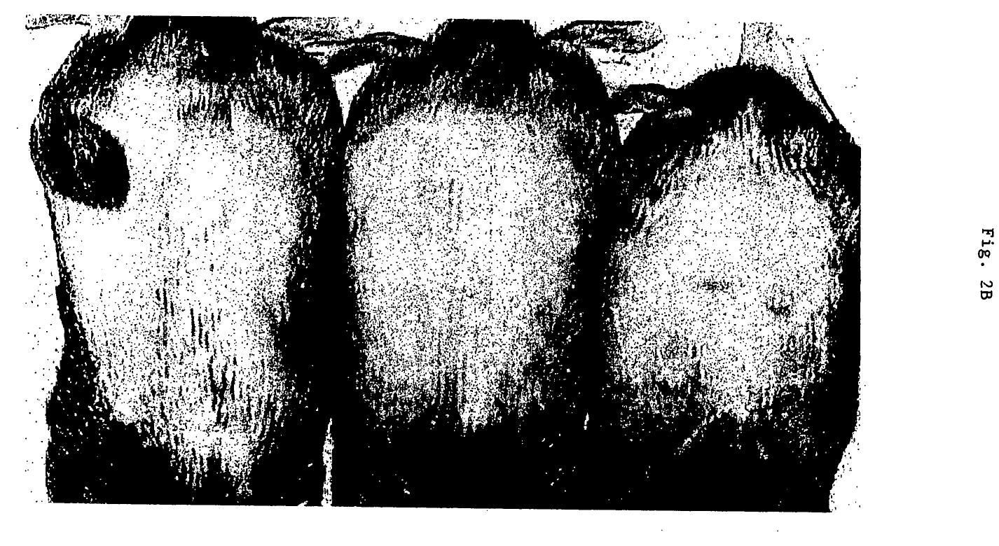

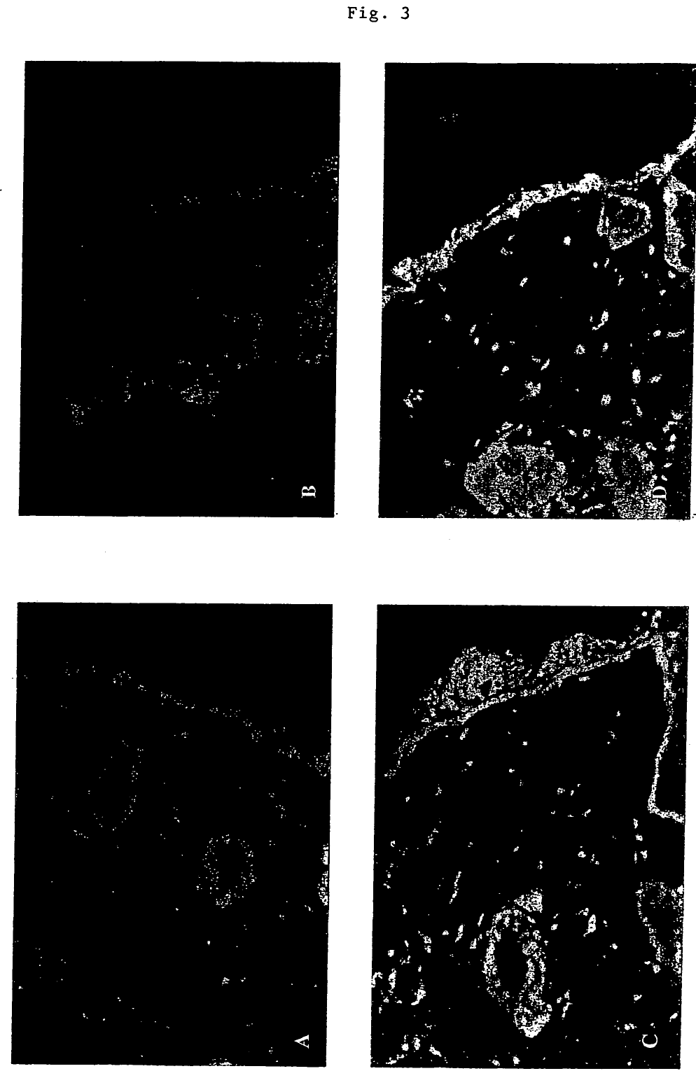

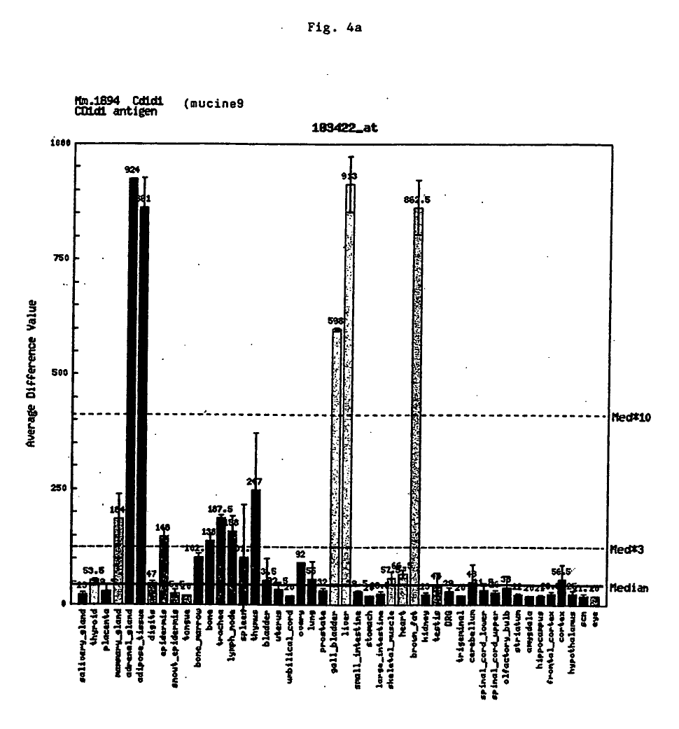

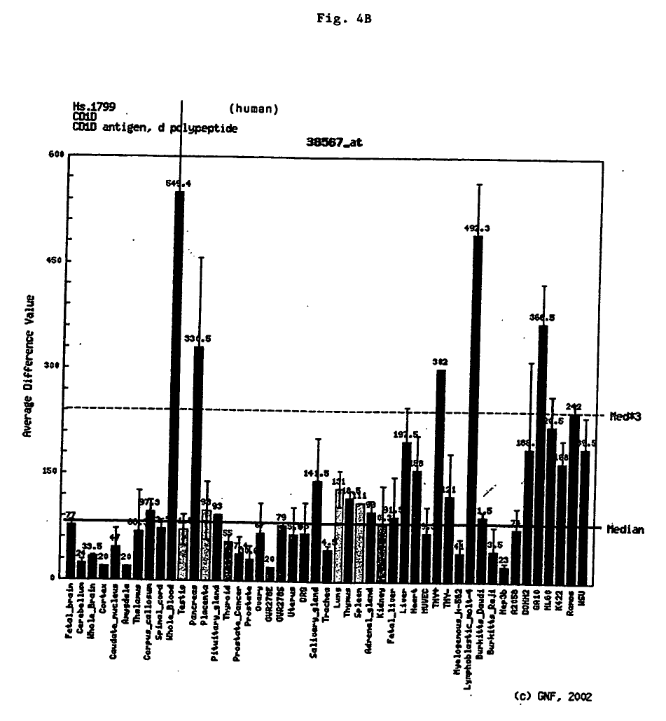

### DETAILED DESCRIPTION OF THE INVENTION

The present invention is essentially based on the finding that CD_{1d}, a transmembrane protein expressed by a number of different cells, in particular epithelial cells, modulates a variety of different responses of the cell to stress. As will become evident from the following detailed description of the preferred embodiments, essentially modifying, specifically blocking the endogenous CD_{1d} function in cells bearing said membrane molecule allows to prevent the detrimental effects of stress, including ultraviolet radiation-induced skin damage, e.g. as a result of burning, epidermal hyperplasia, mutant p53 accumulation, inflammation, immune suppression and skin ageing. Even more surprising is the finding that when essentially blocking CD_{1d} function in epithelial cells induction of cancer in said cells, i.e. basal cell carcinoma, squamous cell carcinoma, malignant melanoma, colon, breast, liver, prostate, kidney, pancreas cancer etc., may be prevented. In addition, it has been surprisingly found that modifying, in particular blocking CD_{1d} function influences hair growth and/or development.

CD_{1d} as such is a type 1 transmembrane MHC class 1 like protein that non-covalently associates with β₂-microglobulin. The CD_{1d} molecule is recognized by a T-cell receptor of natural killer T-cells (NKT) which play a role in immune modulatory and effectory reactions. It has been demonstrated that CD_{1d} may present lipids to NKT cells for their activation, which notion is supported by the CD_{1d} crystal structure having two highly hydrophobic grooves, necessary for presenting hydrophobic molecules such as lipids to the immune system.

In the studies leading to the present invention it has surprisingly been noted that CD_{1d} gene transcription in mouse skin is responsive to external stress, such as UV radiation, which finding has been confirmed in human keratinocytes. In addition it has been noted that skin CD_{1d} mediates UV-induced skin damage/inflammation by inducing COX-2 and TNF-α gene transcription and also inhibiting UV-induced apoptosis.

Without wishing to be bound to any theory it is currently assumed that one of the endogenous tasks of CD_{1d} in living organisms is to directly control normal epithelial cell homeostasis. Normal skin homeostasis is dependent on the critical and fine tuned balance between epidermal differentiation, apoptosis, proliferation and anti-apoptosis of epidermal cells. In the skin, these processes are regulated via lipids, in particular by means of ceramides and glucosylceramides (sphingolipids). While the nucleated cell layers generate glucosylceramides (GlcCer), the proportions of GlcCer to Cer decrease late in epidermal differentiation, with the Cer content peaking in the stratum corneum acting as extracellular constituents of the epidermal permeability barrier. In addition to their structural properties, ceramides are associated with inhibition of cellular proliferation, induction of cellular differentiation and programmed cell death. In contrast, GlcCer induce cell proliferation and inhibit programmed cell death.

Based on the findings in the present invention, CD_{1d} appears to be one of the receptors via which the above mentioned lipids might fulfill their biological task. Specifically, CD_{1d} seems to negatively regulate apoptosis. In consequence, in cells under a stress situation, e.g. when exposed to UV-radiation, CD_{1d} supports a continued existence of said stressed cells, even when their genetic material is damaged and/or mutated, which damaged cells will contribute to inflammation processes induced and eventually account for the phenomenon of ageing or eventually tumour development.

In blocking and/or modifying endogenous CD_{1d} function, apoptosis of cells under stress may be promoted, instead of their survival and propagation, with the effect that cells that have been damaged to a certain extent, particularly at the DNA level, do not have the chance to proliferate and in case disseminate in the body. The cells once dead will then be extinguished by natural processes in the body and be replaced by “healthy” epithelial cells. Likewise, by means of blocking or modifying CD_{1d} also an interaction with NKT is substantially prevented or altered, wherein the phenomenon of immune suppression during exposure to UV radiation will be essentially reduced or barred at all. Also, this condition is supposed to assist the organism's immune system to eradicate damaged cells, brought about by exposure to UV.

The substance capable of blocking and/or modifying the CD_{1d} transmembrane molecule's activity may be any substance interfering with the endogenous biological function of CD_{1d}, and in particular preventing or reducing association of CD_{1d} with endogenous or exogenous lipids. The substances are obtainable by a process comprising the steps of (a) exposing epithelial cells to a substance of interest, (b) subjecting the epithelial cells to a stress situation, (c) determining the effect of said stress to said epithelial cells by screening for one or more of the following assays: (i) epithelial hyperplasia (H&E), (ii) epithelial proliferation (BrUd, PCNA), (iii) epithelial apoptosis, (iv) p53 mutation accumulation, (v) quantitative and qualitative assessment of epithelial lipids, (vi) co-clustering patterns of apoptotic and non-apoptotic cell surface receptors, (vii) production of pro-inflammatory cytokines, (viii) production of immuno-modulatory cytokines, (ix) markers of inflammation, (x) anti-apoptotic transcription factor activity (xi) markers of ageing, and (d) comparing the results obtained with a control. Such a control may e.g. be an assay, wherein the cells have been subjected to the same stress situation, wherein, however, no substance to be investigated had been added (negative control). Likewise a control may also be, including a substance with a known positive effect in the assay and determining the difference in effect achieved by the substance investigated and the known substance (positive control).

A substance is considered to be active in the context of this application, in case it prevents the negative effects of stress as detailed according to any of the above assays.

It will be appreciated that CD_{1d} activity may be blocked and/or modified by substances acting on the genetic level or at the protein level.

Substances acting on the genetic level are compounds influencing, in particular preventing transcription or translation of the CD_{1d} gene, such as polynucleotides anti-sense to at least a part of the CD_{1d} gene or the CD_{1d}-mRNA.

The terms oligonucleotide and polynucleotide, which are interchangeably used herein, include linear oligomers/polymers of natural or modified monomers or linkages, including desoxyribonucleosides, ribonucleosides, α-anomeric forms thereof, polyamide nucleic acids, and the like, capable of specifically binding to the target nucleic acid by way of a regular pattern of monomer-to-monomer interactions, such as Watson-Crick type of base pairing, Hoogsteen or reverse Hoogsteen types of base pairing, or the like. Usually the monomers are linked by phosphodiester bonds or analogs thereof to form oligonucleotides ranging in size from a few monomeric units, e.g. 3-5, to several 100 or even thousands of monomeric units.

The (anti-)sense oligo-/polynucleotides may also contain pendent groups or moieties, to enhance specificity, nuclease resistance, delivery, or other property related to efficacy, such as e.g. cholesterol moieties, duplex intercalators such as acridine, poly-L-lysine, “end capping” with one or more nuclease-resistant linkage groups such as phosphorothioate, and the like. The corresponding oligonucleotide may be used for blocking transcription, RNA processing and/or translation of the mRNA, Consequently, the oligonucleotide may comprise exon, but also intron sequences of the CD_{1d}-target gene, as desired.

The nucleotide sequence of the human CD_{1d} gene or mRNA is obtainable from NCBI (Accession numbers: AP002532 and NM_{—}001766, respectively). Based on his general knowledge and skill, the skilled person may select at least a portion of the coding region of the CD_{1d} gene and design an appropriate anti-sense polynucleotide, that prevents transcription and/or translation of the CD_{1d} gene. Likewise, also a part of the non-coding region of the CD_{1d} gene may serve as an agent for preventing transcription or reducing the number of transcripts, respectively, of the CD_{1d} gene. Here, in particular parts of the promotor region may serve as a template for preparing an antisense polynucleotide, but likewise transitions regions from introns and exons and vice versa. According to a preferred embodiment such a substance may be an DNA or a cRNA (RNA-interference).

Yet, apart from the CD_{1d} gene being the target, also the activity of a number of regulatory molecules which control epithelial homeostasis such as ceramides and/or glucosylceramides, may be modified such, that they exert the desired effect on the CD_{1d} molecule. To this end, the number of the glucosylceramide synthase transcripts may be reduced by designing an polynucleotide antisense to at least a part of the glucosylceramide synthase gene or glucosylceramide synthase mRNA, so that eventually the signal to epithelial cells to proliferate is turned down. The nucleotide sequence of the glucosylceramide synthase gene is disclosed in Ichikawa et al., PNAS 93 (1996), 4638-4643, which document is incorporated herein by way of reference. Likewise, non coding regions may serve as a template for the antisense polynucleotide, such as the promotor region and/or transitions from introns to exons and vice versa. According to a preferred embodiment such a substance may be a DNA or a cRNA (RNA-interference).

Apart from reducing the proliferation signal also the signal driving epithelial cells to apoptosis via the CD_{1d} molecule may be enhanced. In this respect the number of corresponding transcripts may be increased, which may be effected by providing a higher number of polynucleotides encoding a sequence comprised by the sphingomyelinase or ceramide synthase gene and/or the sphingomyelinase or ceramide synthase mRNA.

Apart from the genetic level, the biological activity of the CD_{1d} molecule may also be modified, in particular blocked at the protein level, in particular by any substance binding to the CD_{1d} receptor on or in epithelial cells and blocking the endogenous biological functionality thereof.

According to a preferred embodiment the substance capable of modifying, in particular blocking biological CD_{1d} function is a polypeptide or a peptide, in particular hydrophobic peptides, more preferably an antibody, or a part thereof, that binds to the CD_{1d} receptor and blocks its biological function, such as the interaction with NKT. As parts thereof, in particular mini-antibodies are envisaged lacking the F_{c}-part. According to an alternative embodiment the substance capable of blocking the biological CD_{1d} function may also be a soluble CD_{1d} receptor, that is, that part of the polypeptide lacking the region, anchoring the polypeptide in the membrane. The soluble CD_{1d} receptor will scavenge the in vivo ligands that promote survival of the stressed cells, thus promoting apoptosis. In addition, binding of the natural killer cells to CD_{1d} in vivo will be reduced, thus preventing activation of the T-cells and consequently inflammatory and/or immunosuppressive reactions.

According to a preferred embodiment the substance capable of blocking and/or modifying biological CD_{1d} function is a lipid derived from a plant, microbe or animal, including a phospholipid, ganglioside, sphingolipid, glycosphingolipid, phosphatidylinositol phosphate, sterol, polyphenol, glyceride or fatty acid. These lipids may influence CD_{1d} function by directly binding the CD_{1d} molecule or indirectly by influencing CD_{1d} gene expression.

According to an alternative embodiment the substance capable of blocking and/or modifying biological CD_{1d} function is a ceramide, such as ceramide 8 or sphingosine phosphocholine or a ligand of a receptor belonging to the TNF-superfamily, in particular CD95/APO-1/Fas, which induces apoptosis thus interfering with the anti-apoptotic function of CD_{1d}. In another embodiment the objective substance is an organic compound obtained by chemical synthesis.

It is well established that ceramide glycosylation, via glucosylceramide synthase, and the subsequent build up of glucosylceramides allows cellular escape from stress-induced programmed cell death, conferring cancer cell resistance of a variety of cancers including breast, skin, colon and epitheliod carcinomas, to cytotoxic anti-cancer agents. As CD_{1d} can bind glucosylceramide and is over-expressed by the same multi-drug-resistant cancer cells (e.g squamous cell carcinoma), it is envisioned that the anti-apoptotic activity of CD_{1d} regulates cancer cell resistance to cytotoxic drugs, possibly at the level of protein-glucosylceramide binding. Thus, in principle the substances of the present invention that block and/or modify endogenous CD_{1d} function strongly decrease multi-drug resistance of a variety of cancers including skin, gut and breast cancers.

In principle, the substances of the present invention may also influence the bi-directional trafficking of CD_{1d} to and from the membrane.

The substances may be included in any composition suitable for administering the substance to an individual, in particular a food composition, a cosmetic composition or a pharmaceutical composition.

The pharmaceutical compositions containing at least one of the substances capable of blocking or modifying the CD_{1d} surface molecule according to the invention can be administered for prophylactic and/or therapeutic treatments. In therapeutic applications, compositions are administered to a patient already suffering from a disease, as described herein under, in an amount sufficient to cure or at least partially arrest the symptoms of the disease and its complications. An amount adequate to accomplish this is defined as “a therapeutically effective dose”. Amounts effective for this will depend on the severity of the disease and the weight and general state of the patient.

In prophylactic applications, compositions containing at least one of the substances capable of blocking or modifying the CD_{1d} surface molecule according to the invention are administered to a patient susceptible to or otherwise at risk of a particular disease. Such an amount is defined to be “a prophylactic effective dose”. In this use, the precise amounts again depend on the patient's state of health and weight.

The compounds of the invention are preferably administered with a pharmaceutical acceptable carrier, the nature of the carrier differing with the mode of administration, for example parenteral, intravenous, oral and topical (including ophthalmic) routes.

The desired formulation can be made using a variety of excipients including, for example, pharmaceutical grades of mannitol, lactose, starch, magnesium stearate, sodium saccharin cellulose, magnesium carbonate. This composition may be a tablet, a capsule, a pill, a solution, a suspension, a syrup, a dried oral supplement, a wet oral supplement, dry tube-feeding, wet tube-feeding etc. In order to control the drug release, sustained-release formulations can also be used.

The kind of the carrier/excipient and the amount thereof will depend on the nature of the substance and the mode of drug delivery and/or administration contemplated. E.g., for formulations containing weakly soluble antisense oligonucleotides, micro-emulsions may be employed, for example by using a non-ionic surfactant such as Tween 80 in an amount of about 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences. These various components utilized provide a variety of functions, including regulation of drug concentration, regulation of solubility, chemical stabilization, regulation of viscosity, absorption enhancement, regulation of pH, and the like. For example, in water soluble formulations the pharmaceutical composition preferably includes a buffer such as a phosphate buffer, or other organic acid salts, preferably at a pH of between about 7 and 8.

It will be appreciated that the skilled person will, based on his own knowledge select the appropriate components and galenic form to target the active compound to the tissue of interest, e.g. the colon, stomach, skin, kidney or liver, taking into account the route of administration which may be by way of injection, topical application, intranasal administration, administration by implanted or transdermal sustained release systems, and the like.

The objective substance may also be formulated in a cosmetic product, such as lotions, shampoos, creams, sun-screens, after-sun creams, sun-blocker, anti-ageing creams, ointments and/or anti-hair loss liquids. This proves in particular advantageous for essentially blocking CD_{1d} function in the skin and to prevent the adverse effect of sun radiation, photo-ageing and exposure of the skin to free radicals. Thus, e.g. by providing a sun-screen containing in addition to a common agent, absorbing UV-light a substance as defined herein, a protection to the sun may be provided, which by far exceeds anything known so far. This feature is based in particular on the fact that the objective substance will penetrate the skin and exert its effect after having reached the target molecules. Since this effect will stay for a while, protection to the sun will even be present in case the sun-screen has been rubbed off or has been washed off, as e.g. during sport etc. Yet, apart from sun-screens the objective substances may be included in common day-creams, lotions etc. to prevent negative effects of the daily environment, including pollution, oxidative stress etc. It will be appreciated that the present cosmetic products will contain a mixture of different ingredients known to the skilled person, ensuring a fast penetration of the objective substance into the skin and preventing degradation thereof during storage.

Another high important composition according to the present invention is food material. In our present society a great deal of food is ingested, such as sausages, salted or grilled meat etc., that contains preservatives, ingredients or substances, that are injurious to the gut. E.g. grilled meat contains aliphatic and aromatic compounds known to be cancerogenic. Also preservatives, that kill micro-organisms contained in food material (e.g. sausages) by manipulating their DNA, will exert a similar effect to cells of the gut. In fact, the number of intestinal cancer is steadily increasing in our society, which may be attributed at least in part to the type of food taken by humans.

Consequently, the present invention provides a food composition that prevents the onset and/or development of such gut disorders, such as a composition selected from the group consisting of milk, or fermented milk products, such as e.g. yogurt, curd, cheese, milk based fermented products, ice-creams, milk based powders, infant formulae, cereal products and fermented cereal based products, mineral water, chocolate or pet food containing at least a substance capable of essentially blocking and/or modifying CD_{1d} function. Since the objective compound will be contained in a food material in amounts, that do not affect the original taste thereof, the consumer will not notice any change in the product, but will experience the beneficial effects thereof, namely a protective or even curing effect. Once the food material has been ingested the objective substances will arrive at the target cells, which may be epithelial cells of the gut, i.e. of the stomach or the intestine, and will bind to the CD_{1d} receptor and exert its activity. As a consequence, cells, that are already damaged will preferably go to apoptosis instead of being maintained in said damaged form.

Since epithelial cells bearing CD_{1d} have been found in a number of organs, such as the liver, the small intestine, the colon, the kidney, the prostate, the uterus, the pancreas, breast, skin and conjunctiva, the choice of the composition as detailed above will, by and large depend on the target tissue. As will be understood, for skin a cosmetic product might be the composition of choice, while in case of delivering the objective substance directly to the gut or the colon, a food product may be first choice. However, a food product may also be suitable for delivering the objective substance or substances to other organs, such as the kidney or the liver, which will depend on the stability of the substance in the body and its capacity of being absorbed by the body in the gut. Since food is a daily ingested material such a product offers a great variety of different possibilities. Yet, in case the objective substance is prone to degradation in the gut a pharmaceutical composition may be selected, providing e.g. encapsulation or other galenic forms to deliver the objective substance to the target tissue/to target cells.

It will be understood that the concept of the present invention may likewise be applied as an adjuvant therapy assisting in presently used medications. In this respect the pharmaceutical composition of the present invention may be administered together with e.g. cytostatika so as to prevent escape of the tumor treated from the treatment, which sometimes occurs in long term treatments of certain tumors or to assist in killing residual cancer cells not captured with the pharmaceutical regimen. Since the substance(s) of the present invention may easily be administered together with food material special clinical food may be applied containing a high amount of the objective substances. Also melanoma may be directly treated with an antibody medication against melanoma together with a pharmaceutical composition or a cosmetic product as described herein. It will be clear that on reading the present specification together with the appending claims the skilled person will envisage a variety of different alternatives to the specific embodiments mentioned herein.

In principle, the substances according to the present invention may be used for the treatment and/or prevention of damages in epithelial tissues, such as e.g. in the skin, gut, eye, lung, liver, prostate, breast, kidney and/or in the uterus, which are produced by a stress situation, e.g. by means of a chemical, biological or a physical stress, e.g. by exposure to oxidants or carcinogens, exposure to bacteria, viruses, fungi, lipids derived from surrounding cells and/or microbes, or exposure to UV-irradiation. Likewise, the substances may be utilized for preventing and/or treating hair loss.

Consequently, the substances and/or compositions according to the present invention may be utilized for treating and or preventing damages of the skin, in particular actinic and ageing damages of the skin such as dryness, actinic keratoses, irregular pigmentation (notably comprising freckling, lentigines, guttate hypomelanosis and persistent hyperpigmentation), wrinckling (notably comprising fine surface lines and deep furrows), stellate pseudoscars, elastosis, inelasticity, telangiectasia, venous lakes, purpura, comedones, sebaceous hyperplasia, acrochordon, cherry angiogema, seborrhea keratosis, lentigo, basal cell carcinoma and squamous cell carcinoma, skin burning and/or blistering, cataract formation, epidermal hyperplasia, inflammation, immune suppression, and cancer, e.g. non-melanoma and melanoma skin cancers.

In order to arrive at additional substances having the above characteristics the present invention also provides a method for screening for such substances. In this method epithelial cells are utilized that may be in the form of a primary culture, i.e. directly derived from an individual or in the form of a cell line. For carrying out the method a cell culture is particularly preferred, since it allows for the continuous supply of epithelial cells during the experiments. Care must be taken that the cell culture of epithelial cells used exhibit the same phenotypic traits as do cells of a primary culture or epithelial cells directly obtained from a tissue sample. It will be understood that the person skilled in the art will select the starting material depending on the assay. Hence, if a first round assay is to be carried out a cell culture design seems to be most appropriate, while in case for further rounds, i.e. assessing the activity of potential candidates, the tissue or even the animal model seems to be more appropriate.

The epithelial cells are exposed to a substance of interest for a time period sufficient to ensure a contact of the substance with the cells. In a next step the epithelial cells are exposed to a stress situation, which may be effected e.g. by irradiating the cells with different dosages of UV light, or adding hydrogen peroxide or toxic chemicals to the cell culture. However, the type of stress is not critical as long as the cells are challenged to initiate processes, normally started under stress situations, such as e.g. the production of pro-inflammatory cytokines e.g. IFN-g, TNF-a, IL-1, IL-6, IL-8, apoptosis, altered lipid metabolism, increased production of p53, altered cell signaling as a result of altered patterns of cell surface receptor co-clustering, NF-κB activation, AP1 activation, showing hyperproliferation (anti-apoptosis), altered barrier function etc. It will be understood that also more than one substance may be tested at the same time, that is a cocktail of one or more substances, which might prove beneficial for the second or further round of assaying.

In a next step the effect of said stress on the epithelial cells is determined by assessing one or more of the following features, for example: epithelial proliferation (PCNA: Ouhtit et al., American Journal of Pathology [2000], 156: 201-207; BrUd: Lu Y-P et al., Cancer Research [1999], 59: 4591-4602); epithelial apoptosis (Tunel Assay; modification of protocol outlined by Ouhtit et al., American Journal of Pathology [2000], 156: 201-207); p53 mutation accumulation (Allele-specific polymerase chain reaction [AS-PCR] and single-strand conformation polymorphism [SSCP], Ananthaswamy et al., Nature Medicine [1997], 3: 510-514); production of pro-inflammatory and immuno-modulatory cytokines (e.g. TNF-α, PGE-2, IL-1, IL-6, IL-8, IL-4, IL-10, Platelet Activating Factor, TGFβ); markers of inflammation (e.g. COX-2, iNos); and anti-apoptotic transcription factor (including AP-1, NFkappaB) activity by TaqMan Real-time RT-PCR, ELISA, and Immunohistochemistry; qualitative and quantitative assessment of phospholipids, glycosphingolipid and sphingolipid content (Electron-Spray Tandem Mass Spectrometry); analysis of co-clustering patterns of epithelial cell surface receptor molecules including cytokine receptors (e.g. IL-6), molecules of the TNF-superfamily of receptors (e.g. CD95/APO-1/Fas) and growth regulating receptors (e.g. EGF, Insulin) by fluorescence resonance electron transfer analysis (FRET); markers of ageing, e.g. elastases, collagenases, metalloproteinases, gelatinase, stromelysins, telomerase.

The results obtained are then compared with a control, which may simply be an assay, wherein the same type of cells are exposed to the same stress conditions with the proviso, that no compound to be assessed for its CD_{1d} blocking capacity is provided. As for the animal model a positive control is represented by a CD_{1d}^{-/-} animal, wherein CD_{1d} activity is lacking at all.

The following examples illustrate the invention in more detail without restricting the same thereto.

### EXAMPLE 1

Generation of CD_{1d} Mutant Mice

Mouse CD_{1d} is encoded by two genes, CD_{1d1} and CD_{1d2}, that share a high degree of nucleotide sequence identity (Bradbury et al., EMBO J., 7 (1988), 3081-3086). The product of the CD_{1d1} gene is recognized by all anti-CD₁ antibodies that have been described, whereas surface expression of the CD_{1d2} product has not yet been demonstrated. In addition, the predicted α2 domain of the CD_{1d2} gene product lacks an intra-domain disulfide bond that is found in the α2 domain of all published classic and non-classic MHC class I molecules (Bradbury, supra). This disulphide bond is thought to be critical for the folding of the antigen-binding groove. Thus, the CD_{1d2} gene may not encode a functional antigen-presenting molecule, and all functions previously attributed to mouse CD₁ may be effected by the product of the CD_{1d1} gene. For this reason, it was decided to introduce a targeted mutation into the CD_{1d1} gene, while leaving CD_{1d2} intact.

The CD_{1d} gene was isolated from a strain 129/Sv phage library with a probe generated by polymerase chain reaction. The targeting construct was prepared using a 2.8 kb Apa1 fragment containing the 5′ region of the CD_{1d} gene, a 3.2 kb BamHI-NotI fragment containing the 3′ region of the CD_{1d} gene (the NotI site in this fragment comes from the pBluescript vector into which phage DNA was initially subcloned), a neomycin resistance gene (neo), and the pBluescript plasmid (Stratagene). This construct was designed to delete a fragment of about 200 bp from the exon encoding the α2 domain of CD_{1d1}. The strain 129/Sv-derived embryonic stem (ES) cell line TL1 was transfected with the NotI-linearized targeting vector. G418-resistant colonies were selected and isolated as described in Van Kaer et al., Cell 71 (1992), 1205-1214. Genomic DNA from individual clones was digested with EcoRI and hybridized with a 2.3 kb CIaI-EcoRI probe from the 5′ end of the CD_{1d1} gene. Recombination was confirmed by digestion with KpnI and hybridization with a 700 bp BamHI-EcoRI probe from the 3′ end of the CD_{1d1} gene. Chimeric mice were mated with C57BL/6 mice to score for germline transmission, and heterozygous mutant mice were intercrossed to obtain (C57BL/6×129/Sv) F2 homozygous mutants. Mice were typed for their CD_{1d1} status by genomic southern blotting with the 5′ probe. Mutant mice were healthy and bred normally.

Because the ES cells and mouse strain used to generate mutant animals differ in their TL status (129/Sv is a TL+ stain and C57BL/6 is a TL−strain) all mice used in this study were genotyped for TL. To type mice for their TL status, tail DNA was digested with BglII and hybridized with a TL-specific probe that detects a polymorphism between strains 129/Sv (TL+) and C57BL/6 (TL−) (Pontarotti et al., Proc. Natl. Acad. Sci. USA 83 (1986), 1782-1786). This probe was generated by polymerase chain reaction using a set of primers designed on the basis of published sequences (Pontarotti, supra):

5′-TATACAGAGCTCCGTAGGAC-3′;
and
5′-AGTTGTCTGCAGCCACGAAC-3′.

The CD_{1d1} mutant and wild-type mice were housed in a specific-pathogen-free barrier animal facility, accredited by the American Association for Accreditation of Laboratory Animal Care (AAALAC). Animals were used between 12-16 weeks of age at the start of the experiments. They were housed in filter-protected cages with a 12 h light-dark controlled cycle, and provided with autoclaved NIH open formula mouse chow and water ad libidum. The institutional Animal Care and Use Committee approved all procedures. Within each experiment all mice were aged- and sex-matched.

It is underlined that other genetic backgrounds can be used for creating a CD_{1d} mutant mouse, such as Balb/C genetic background.

### EXAMPLE 2

UV Irradiation of Mice

A bank of five Philips TL-40W/12 sunlamps (Philips, The Netherlands) was used to irradiate the mice. These lamps emit a spectrum from 270 to 400 nm; 54% of the irradiation was within the UVB range (280-315 nm) of the solar spectrum, with 45% being in the UVA (315-400 nm) region and less than 1% in the UV-C (240-280 nm) range. The irradiance of the five bulbs averaged 10 W/m², as measured by a UVB PMA research radiometer.

The dorsal hair of the mice was removed with electric clippers and the mice were placed into a plexiglass box separated into individual compartments by Plexiglas dividers and covered with a wire top which decreased the incident dose by 14%. For each UV-irradiation, the box was placed each time in the same position under the lamps to compensate for the uneven distribution of energy along the length of the bulbs. The mice were exposed once or twice to an incident dose of 86 mJ/cm² UVB from five Philips TL-40 W/12 sunlamps. Mice were exposed to a second dose of UVB radiation 96 h after the first exposure. All mice were analyzed for signs of skin damage 24, 48, 72 and 96 h after their last UVB exposure.

Visually, a clear difference in the degree of skin damage was observed between wild-type and CD_{1d} knockout mice following UVB-irradiation of their shaved dorsums. Whilst clear and significant skin damage (burning, skin lesions) was exhibited by UV-irradiated wild-type mice, no obvious signs of skin damage were detected in UV-irradiated CD1d knockout mice.

### EXAMPLE 3

Measurement of Apoptosis in Epidermis

Apoptotic cell death was detected using the DeadEnd™ Fluorometric TUNEL System (Promega) which measures the fragmented DNA of apoptotic cells by catalytically incorporating fluorescein-12-dUTP at 3′-OH DNA ends using the enzyme Terminal Deoxynucleotidyl Transferase (TdT). TdT forms a polymeric tail using the principle of the TUNEL assay. Briefly, formaline fixed paraffin embedded tissue sections on slides were deparaffinized twice in fresh xylene for 5 min at room temperature. They were washed in 100% ethanol for 5 min and then rehydrated sequentially by immersing the sections through graded (100%, 95%, 85%, 70%, 50%) ethanol washes for 3 min. Afterwards, the sections were immersed in 0.85% NaCl for 5 min, washed in PBS and then fixed in 4% paraformaldehyde for 15 min followed by two washes in PBS. After removing residual fluid from the sections by tapping, each tissue section was covered with 20 μg/ml proteinase K (Sigma) for 8-10 min at room temperature. After proteinase K treatment, tissue sections were rinsed in PBS and then fixed by immersing in 4% paraformaldehyde for 5 min. This was followed by a wash in PBS, removal of residual fluid by tapping and incubation of the sections in equilibrium buffer (Promega) for 5-10 min. After equilibration, the sections were incubated in a humidified chamber with TdT enzyme for 1 h at 37° C. Sections were soaked in stop buffer (SSC; Promega) for 15 min to terminate the reactions and then rinsed in three changes of PBS. After rinsing, sections were stained with propidium iodide solution freshly diluted to 1 μg/ml in PBS for 15 min in the dark. They were then washed three times in deionized water for 5 min, and afterwards, excess fluid wiped off the area surrounding the cells. The sections were then immediately examined under a fluorescence microscope.

At 2, 6, 24, 48, 72 and 96 h after UV exposure (acute/chronic) a TUNEL Assay (modification of protocol outlined by Ouhtit et al., American Journal of Pathology [2000], 156: 201-207), of the skin taken from CD_{1d}^{-/-} and wild-type mice was carried out. The results revealed that epidermal cells within the skin of CD_{1d}^{-/-} mice were undergoing a high degree of apoptosis compared to wild-type mice. In contrast, in wild-type skin the epidermal cells were undergoing significantly less apoptosis.

### EXAMPLE 4

Measurement of Epidermal Hyperplasia

Dorsal skin biopsies were fixed overnight in 4% paraformaldehyde and paraffin embedded. Sections were stained with hematoxylin and eosin (H&E) and viewed by light microscopy.

Following UVB exposure, CD_{1d}^{-/-} mice exhibited significantly reduced epidermal hyperplasia 48 h after the last UVB treatment compared to UV-irradiated wild-type mice.

### EXAMPLE 5

Gene Profiling

In order to elucidate CD_{1d} function a gene profiling assay comparing wild-type and CD_{1d} knockout mouse gene expression had been performed.

Skin tissue was extracted from 5 individual wild-type and CD_{1d} knockout mice and extracted separately using Trizol kit (Invitrogen AG, Basel, Switzerland) and then Qiagen RNeasy mini-kits (Basel Switzerland) according to manufacturer instructions with DNase I treatment to remove any genomic DNA contamination. RNA samples were quantified by OD then analyzed via dynamic gel electrophoresis with the Agilent Bioanalyser for intact 28S and 18S rRNA (All 28/18 ratio's were between 1.6 and 2.0). Study samples were judged to contain sufficient amounts of high-quality RNA for hybridization to GeneChips. As another quality control measure, prior to hybridization with Affymetrix GeneChips (Affymetrix, Inc., Santa Clara, Calif.), we confirmed that all samples gave strong signals for pre-selected genes, using Affymetrix test chips (Test chip 5′/3′ ratios were less than 3.0).

For skin, 10 μg total RNA was the starting material for all individual mouse samples. In general, total RNA was converted to biotinylated cRNA, hybridized in the Affymetrix probe array cartridge, stained, and then quantified. First and second strand cDNA synthesis was performed using the SuperScript Choice System (Invitrogen AG, Basel, Switzerland), according to manufacturer instructions, but using an oligo-dT primer containing a T7 RNA polymerase binding site. Labeled cRNA was prepared with the RNA Transcript Labeling kit (Enzo Biochem Inc., NY). Biotinylated CTP and UTP were used together with unlabeled NTPs in the reaction, and unincorporated nucleotides were removed with Nucleospin columns (Macherey-Nagel, Düren, Germany).

cRNA (20 μg) was fragmented at 94° C. for 35 min in buffer containing 200 mM Tris-acetate pH 8.1, 500 mM KOAc, 150 mM MgOAc. Prior to hybridization, fragmented cRNA in hybridization mix (Buffer containing 100 mM MES, 1M NaCl, 20 mM EDTA, 0.01% Tween 20, 0.5 ng/μl BSA, 0.1 ng/μl herring sperm and Affymetrix controls), was heated to 95° C. for 5 min, cooled to 45° C. and loaded onto an Affymetrix probe array cartridge. The probe array was incubated for 16 h at 45° C. at constant rotation (60 rpm), then exposed to Affymetrix washing and staining protocol.

This protocol included:
one wash with non-stringent buffer (6× SSPE, 0.01% Tween 20, 0.005% antifoam)
one wash with stringent buffer (100 mM MES, 0.1 M NaCl, 0.01% tween 20)
First stain with 0.01 mg/ml streptavidin-phycoerythrin conjugate (Molecular Probes) in buffer containing 100 mM MES, 1M NaCl, 0.05% Tween 20, 4 mg/ml of BSA.
one wash with non-stringent buffer (6× SSPE, 0.01% Tween 20, 0.005% antifoam)
Second stain with 3 μg/ml of biotinylated anti-streptavidin +0.2 mg/ml of IgG in buffer containing 100 mM MES, 1M NaCl, 0.05% Tween 20, 4 mg/ml of BSA.
Third stain with 0.01 mg/ml streptavidin-phycoerythrin conjugate (Molecular Probes) in buffer containing 100 mM MES, 1M NaCl, 0.05% Tween 20, 4 mg/ml of BSA.
one wash with non-stringent buffer (6× SSPE, 0.01% Tween 20, 0.005% antifoam)

A mathematical method was developed and applied to the raw GeneChip data for the selection of differentially regulated genes. This method moves beyond setting a single fold change cut-off by considering the standard deviation (SD) in the context of absolute expression, or absolute difference intensity (ADI).

The method include the following steps: (A) data processing by the commercially available “MAS5” Affymetrix program (Santa Clara, Calif., USA) and rescaling, (B) logarithmic transformation to distribution normality of the rescaled data, (C) multiple hypotheses (one per gene) analysis of variance (ANOVA) testing, (D) the determination of the robust mean within condition SD (equation 1), within bins of 200 genes ordered by mean ADI levels, to determine a significance limit SD between condition, named the REGExpress function (equation 2 from Genome Biology 2001 2(12): preprint0009.1-0009.31); and (E) subsequent ranking of genes by the p value of the REGExpress and ANOVA, to help focus at effect importance. The selection is made with the p value resulting from multiple hypotheses (one per gene) ANOVA testing and/or with the p value resulting from REGExpress.

Probe arrays were scanned at 488 nm using an Argon-ion Laser (made for Affymetrix by Agilent). Readings from the quantitative scanning were analyzed with Affymetrix Gene Expression Analysis Software.

The findings are summarized in the tables I to III below. The fold increase (+) or decrease (−) is the statistically significant relative fold increase or decrease of a gene expressed in CD1d knockout mice compared to the same gene expressed in wild-type mice. It becomes clearly evident that blocking CD1d upregulates genes controlling hair follicle development, and down-regulates genes involved in inflammation and cancer development.

TABLE I
Genes which regulate hair follicle development

| | Fold | | | |
|---|---|---|---|---|
| | increase/ | Mean | Mean | Biological |
| Gene Name | decrease | Wt | CD1d−/− | Function |
| mmu-crystalline | +27.0 | 0.922 | 4.251 | thyroid binding |
| | | | | protein regulating |
| | | | | hair follicle |
| | | | | development |
| PPatched homolog 2 | +2.763 | 3.810 | 4.826 | hair follicle |
| | | | | development |

TABLE II
Genes which regulate inflammation

| | Fold | | | | |
|---|---|---|---|---|---|
| | increase/ | Mean | Mean | | |
| Gene Name | decrease | Wt | CD1d−/− | Biological Function | Disease Association |
| TGF beta | +1.743 | 3.630 | 4.186 | Signalling molecule of | |
| activated Kinease | | | | the p38-MAPKinase and the | |
| | | | | Stress activated protein | |
| | | | | Kinase (SAPK) pathways. | |
| Rel-A(NfKappaB) | −0.735 | 7.350 | 7.043 | anti-apoptotic, induction | inflammatory disorders |
| | | | | of inflammatory cytokines. | |
| Cytochrome beta | −0.738 | 6.829 | 6.525 | superoxide generation | |
| Plasminogen activator | −0.357 | 3.906 | 2.875 | Serine protease inhibitor. | inflammatory disorders |
| inhibitor (PAI-1) | | | | Regulates fibrolysis. | |
| MRP14 | −0.202 | 3.943 | 2.344 | Ca⁺⁺ dependent regulatory | acute and chronic |
| | | | | protein in inflammatory | inflammatory responses |
| | | | | responses. | e.g. Psoriasis |
| Mast cell protease | −0.661 | 6.427 | 6.014 | proteolysis and peptidolysis. | inflammation |
| P-Selectin | −0.685 | 6.439 | 6.060 | Cell adhesion | inflammation |
| TFII-1 | +1.373 | 5.707 | 6.024 | Transcription factor which | |
| | | | | regulates c-Fos activity | |
| Interleukin-6 | −0.268 | 1.882 | 0.565 | cytokine: multi-functional | inflammation |

TABLE III
Genes which regulate cancer growth/development:

| | Fold | | | | |
|---|---|---|---|---|---|
| | increase/ | Mean | Mean | | |
| Gene Name | decrease | Wt | CD1d−/− | Biological Function | Disease Association |
| v-Rel(NfKappaB) | −0.735 | 7.350 | 7.043 | oncogenic- | cancer |
| | | | | transforms cells | |
| Plasminogen activator | −0.357 | 3.906 | 2.875 | serine protease | metastatic tumors |
| inhibitor (PAI-1) | | | | inhibitor | |
| P-Selectin | −0.685 | 6.439 | 6.060 | adherence | facilitates tumor |
| | | | | | metastasis. |
| Cathepsin S | −0.697 | 7.545 | 7.184 | Cysteine protease | malignancy |
| Proliferin | −0.234 | 1.870 | 0.419 | regulates | mouse fibrosarcomas |
| | | | | angiogenesis | |
| Interleukin-6 | −0.268 | 1.882 | 0.565 | | secreted by basal |
| | | | | | cell carcinomas and |
| | | | | | malignant melanomas. |
| CSF-1 receptor | −0.750 | 7.196 | 6.909 | Growth factor | Cancer |
| | | | | regulating cell | |
| | | | | proliferation. | |

### EXAMPLE 6

### Evaluation of the Inflammatory Response Induced by a Single Topical Administration of TPA

Phorbol-12-myristate-13-acetate (TPA) provided by Sigma Aldrich (L'Isle d'Abeau Chesnes BP701, 38297 Saint Quentin Fallavier, France) is dissolved in acetone at the dose of 0.01% (W/V) and 20 μl of the solution is applied topically onto the internal face of the right ear of CD_{1d}^{-/-} mice or wild-type mice in order to induce an acute inflammatory response.

The animals are maintained in individual cages with a standard pellet diet in an animal room with a 12-hour light-dark cycle. The facilities provide a filtered air with a temperature of 22+/−2° C. and a relative humidity of 55+/−10%.

The inflammatory response is quantified 6 hours, 24 hours and 48 hours after application by measuring the ear oedema using a micrometer (<(<<oditest >> provided by Kroeplin Gmbh, Postfach 1255 D36372 Schlüchlern, Germany).

The oedema is calculated as follows:

(oedema=ear thickness of the treated group−ear thickness of the acetone group).

The mean value of CD_{1d}^{-/-} group is compared to the mean value of the wild-type group using the Student's t-test.

### EXAMPLE 7

### Evaluation of the Inflammatory Response Induced by a Single Topical Administration of Arachidonic Acid

Arachidonic acid (5-8-11-eicosatetraenoic acid) provided by Sigma Aldrich (L'Isle d'Abeau Chesnes BP701, 38297 Saint Quentin Fallavier, France) is dissolved in acetone at the concentration of 140 nM and 25 μl of the solution is applied topically onto the internal face of the right ear of CD_{1d}^{-/-} mice or wild-type mice in order to induce an acute inflammatory response.

The animals are maintained in individual cages with a standard pellet diet in an animal room with a 12-hour light-dark cycle. The facilities provide a filtered air with a temperature of 22+/−2° C. and a relative humidity of 55+/−10%.

The inflammatory response is quantified 1 hour, 2 hours, and 4 hours after application by measuring the ear oedema using a micrometer (<<oditest >> provided by Kroeplin Gmbh, Postfach 1255 D36372 Schlüchlern, Germany).

The Oedema is Calculated as Follows:

(oedema=ear thickness of the treated group−ear thickness of the acetone group).

The mean value of CD_{1d}^{-/-} group is compared to the mean value of the wild-type group using the Student's t-test.

### EXAMPLE 8

Evaluation of the DTH (Delayed-Type Hypersensivity) Reaction Induced by Oxazolone

Oxazolone (4-ethyoxymethylene-2-phenyl-oxazol-5-one) provided by Sigma Aldrich (L'Isle d'Abeau Chesnes BP701, 38297 Saint Quentin Fallavier, France) is dissolved in acetone at the concentration of 1% (WN) and 50 μl of the solution is applied once daily for 4 days on the abdominal skin of shaved CD_{1d}^{-/-} mice or shaved wild-type mice.

4 days later the animals are challenged by a single administration (20 μl) onto the internal face of the right ear of oxazolone dissolved in acetone at the dose of 0.3%. The post-challenge response is quantified 24 hours and 48 hours after application by measuring the ear oedema using a micrometer (<<oditest>> provided by Kroeplin Gmbh, Postfach 1255 D36372 Schlüchlern, Germany).

The Oedema is Calculated as Follow:

(oedema=ear thickness of the treated group−ear thickness of the acetone group).

The mean value of CD_{1d}^{-/-} group is compared to the mean value of the wild-type group using the Student's t-test.

### EXAMPLE 9

Evaluation of Skin Damages Induced by UV Irradiation Using a Solar Simulator

A solar simulator (Oriel 81050) equipped with an UVC filter is used to irradiate CD_{1d}^{-/-} mice or wild-type mice.

Irradiation: UVB+UVA doses and to be precised

Effect on epidermis: SBC counts, epidermal hyperplasia measurement

Effect on the dermis: MMP1 and MMP3 expression with immuno-histochemical methods

### EXAMPLE 10

### Regulation of CD_{1d} Gene Transcription by UV Radiation and Role of CD_{1d} in Regulating UV-Induced COX-2 and TNF-α Gene Transcription

Mice

Specific pathogen-free male outbred 129/C57BL/6 wild-type and 129/C57BL/6 CD_{1d} knockout mice were obtained from L. Van Kaer, Vanderbilt University Medical Center (Nashville, TN, USA). The animals were maintained in facilities in accordance with current Swiss regulations and standards. They were housed in filter-protected cages, and ambient lighting was controlled to provide 12 h light/12 h dark cycles. Autoclaved open-formula mouse chow and water were provided ad libidum. All animal procedures were reviewed and approved by the Institutional Animal care and Use Committee. Within each experiment all the mice were matched for age and sex. The mice were 16 weeks at the start of each experiment.

UV Light Source

The UVB source was a bank of five Philips TL-40 W/12 sunlamps (Philips, The Netherlands). These lamps emit a spectrum from 270 to 400 nm; 54% of the irradiation was within the UVB range (280-315 nm) of the solar spectrum, 45% in the UVA (315-400 nm) region and less than 1% in the UV-C (240-280 μm) range. The irradiance of the five bulbs averaged 10 W/m², as measured by a UVB PMA research radiometer. Solar simulated light (UVA+UVB) was produced by a 1000 W exon UV solar simulator (Solar Light Company, PA, USA) equipped with a WG-320 atmospheric attenuation filter (1 mm thick), a visible/infrared band pass blocking filter (UG-5; 5 mm thick), and a dichroic mirror to further reduce visible and infrared energy.

UV Irradiation of Mice

The dorsal hair of the mice was removed with electric clippers. For mice being exposed to UVB radiation they were placed into a Plexiglass box separated into individual compartments by Plexiglass dividers and covered with a wire top which decreased the incident dose by 14%. For each UVB-irradiation, the box was placed each time in the same position under the lamps to compensate for the uneven distribution of energy along the length of the bulbs. For mice being exposed to solar UV radiation (UVA+UVB) the mice were anaesthetized to immobilize them prior to being exposed to the beam of the solar simulator. The mice were exposed once to an incident dose of 86, 215 or 430 mJ/cm² UVB from five Philips TL-40 W/12 sunlamps. Mice exposed to solar light (UVA+UVB) were exposed once to an incident dose of 1680 (1 min), 16,800 (10 min) or 33,600 mJ/cm² (20 min) solar radiation.

UV Irradiation of Keratinocyte Cell Cultures

Confluent cultures of keratinocytes grown in sterile 6-well plates (Corning, Netherlands) were submitted to a single dose (570 mJ/cm²) of solar UV irradiation. Treatment was performed without plastic lids after having removed medium and replaced it by sterile HBSS. Control cultures were not irradiated. After UV exposure, HBSS was removed and medium put back on cultures. The cells were incubated at 37° C. with 5% CO₂ and at various time points thereafter harvested for RNA.

Immunostaining of Mouse Tissue

Biopsies of wild-type mouse skin were fixed in formaline before being embedded in paraffin. Cross sections (5 μm thick) of paraffin embedded tissues were made, deparaffinized by gentle heating, de-hydrated and rehydrated using the following procedure: 2 times for 3 min in Xylol, 3 min in Ethanol 100%, 3 min in Ethanol 95%, 3 min in Ethanol 80% and 3 min in PBS IX. Fresh skin was also embedded in Tissue-Tek (4583, Sakura Finetek, Torrance, USA) and frozen in liquid nitrogen. The sections were then rehydrated in PBS 1× for few minutes. Sections were stained using the anti-mouse CD1d 1H1 primary mAb and developed using the mouse Histostain-plus kit (ZYMED Laboratories Inc., San Francisco, USA).

Extraction of Total RNA from Skin or Cell Lysates

Treated and control cultures of human keratinocytes grown in 6-well plates were placed on ice and washed twice using PBS 1× at 4° C. Cell lysate was obtained by scraping the cells in 350 μl of lysis buffer RLT (74104, Qiagen AG, Basel, Switzerland) supplemented with 1% of β-mercaptoethanol and by briefly vortexing them. QIAshredder columns (79656, Qiagen AG, Basel, Switzerland) were used to homogenize cell extracts by centrifugation at 13,000×g for 2 min. Total RNA was then prepared using RNAeasy kits (74104, Qiagen AG, Basel, Switzerland) according to the manufacturer's protocols. Genomic DNA contamination was removed with on-column DNase digestion using a RNase-free DNase Set (79254, Qiagen AG, Basel, Switzerland). Skin samples of 1 cm×1 cm were cut into small pieces and homogenized in 1 ml of TRIZOL Reagent (15596-026, Invitrogen AG, Basel, Switzerland) using a rotor-stator homogenizer (Polytron, Kinematica, Luzern, Switzerland). The supernatant obtained after centrifugation at 12,000×g was recovered in a fresh tube and incubated for 5 min at room temperature. 0.2 ml of chloroform was added to the tube, which was vigorously shaken for 15 sec and incubated at room temperature for 2-3 min. Samples were centrifuged at 12,000×g for 15 min at 4° C. The upper aqueous phase was transferred to a fresh tube and total RNA precipitated using 0.5 ml of isopropyl alcohol for 10 min at room temperature. The RNA pellet obtained by centrifugation at 12,000×g for 10 min at 4° C. was washed with 1 ml of 75% EtOH followed by centrifugation at 7,500×g for 5 min at 4° C. The RNA pellet was finally dried at room temperature and dissolved in 40 μl of RNase free water by incubating the samples 10 min at 55-60° C. Possible DNA contamination was removed with on-column DNase digestion using a RNase-free DNase Set.

Semi-Quantitative RT-PCR

A. Reverse Transcription-Polymerase Chain Reaction and PCR Reaction

5 μg of total RNA were reverse transcribed by oligo-dT priming to first strand cDNA using the Superscript First-Strand Synthesis System for RT-PCR (11904-018, Invitrogen AG, Basel, Switzerland) according to the manufacturer's instructions. PCR of cDNA was performed to either detect specific expression of a single gene (single PCR) or multiple genes (Multiple gene PCR). For single PCR, 48111 of PCR master mix containing 5 μl PCR Buffer, 31 μl 25 mM MgCl₂, 1 μg 10 mMdNTPs, 0.5 μl 50 μM of both sense and anti-sense oligonucleotides, 3 μl DMSO, 34.51 μl of water and 0.5 μl of 5 U/μl Taq DNA Polymerase was added to 2 μl of cDNA. All the reagents were purchased from Invitrogen (15558-026 and 18427-013, Basel, Switzerland). The number of cycles and the annealing temperature applied to amplify cDNA samples were specific to each gene tested, one cycle consisting of 30 s at 94° C., 30 s at x° C. and 30 s at 72° C., each amplification being preceded by 2 min at 94° C. and finished by 3 min at 72° C. Kit MP-70211 (Maxim Biotechnologies, San Francisco, USA) for multiple gene PCR of genes implicated in apoptosis and inflammation were used as instructed. Prior to using each kit, the condition for running multiple reverse and forward primers at the same time to detect multiple genes was determined. The conditions for using kit MP-70211 which contained primers for detecting TNF-α and COX-2 genes, were as follows: 96° C. for 1 min and 60° C. for 4 min (cycles 2×); 94° C. for 1 min and 60° C. for 2 min (cycles 29×); 70° C. for 10 min (cycle 1×) and 25° C. soak. The DNA sequence of the reverse and forward primers used the MPCR kit for detecting multiple genes under one set of conditions were proprietary and thus are not described in this report.

Primer Sequences and Number of Cycles of Amplification

| | | | Annealing | No. of | Product |
|---|---|---|---|---|---|
| Gene | Primer | Sequence (5′-3′) | temper. | cycles | Size |
| GAPDH | sense | AAT CCC ATC ACC ATC TTC CA | 52 | 16 | 558 |
| | antisense | GTC ATC ATA TTT GGC AGG TT | | | |
| CD1d | sense | GCT CAA CCA GGA CAA GTG GAC GAG | 66 | 27 | 452 |
| | antisense | AGG AAC AGC AAG CAC GCC AGG ACT | | | |

Mouse:

| | | | Annealing | No. of | Product |
|---|---|---|---|---|---|
| Gene | Primer | Sequence (5′-3′) | temper. | cycles | Size |
| GAPDH | sense | TTC ACC ACC ATG GAG AAG GC | 60 | 22 | 236 |
| | antisense | GGC ATG GAC TGT GGT CAT GA | | | |
| CD1d.1 | sense | ACG TCC TGG CAG ACA GTC CCA GG | 60 | 24 | 706 |
| | antisense | TTA ATG TTG AAA AGA GCG TAC TGG C | | | |

B. Relative Quantification of mRNA Levels

Amplification of the genes was analyzed by loading 10 μl of the PCR products on a 3% agarose gel which was run in a 1×TAE buffer containing 2% Ethidium Bromide at 150V for 30 min. The PCR products were visualized as fluorescent bands under UV light. Gels were scanned using a Kodac DC 120 Camera and fluorescence intensity of the bands was quantified using the Software Scion Image β 4.02 Win (Scion Corporation, Maryland, USA).

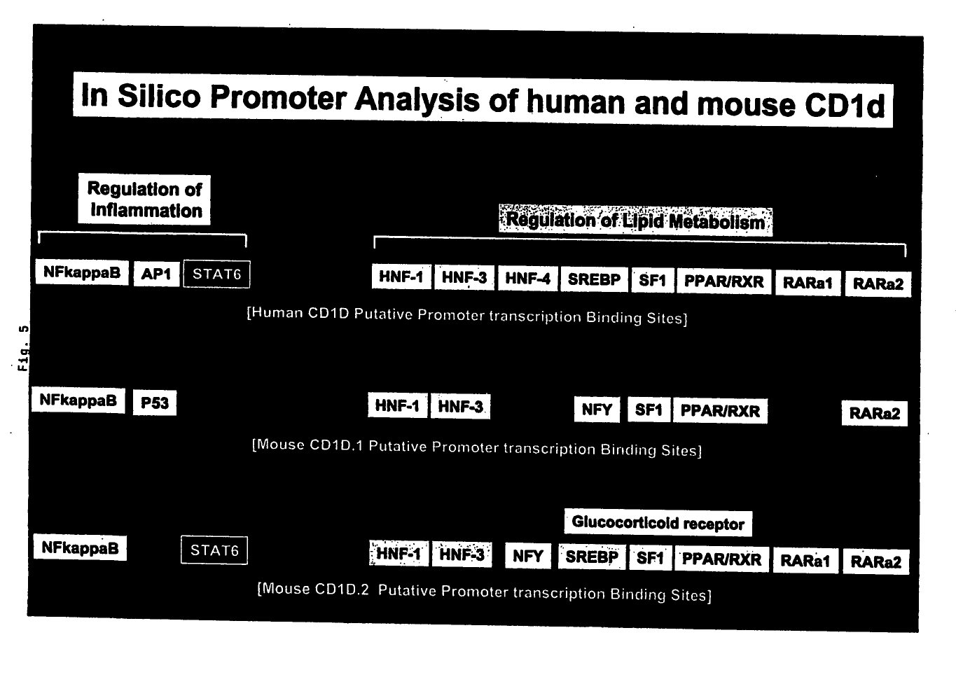

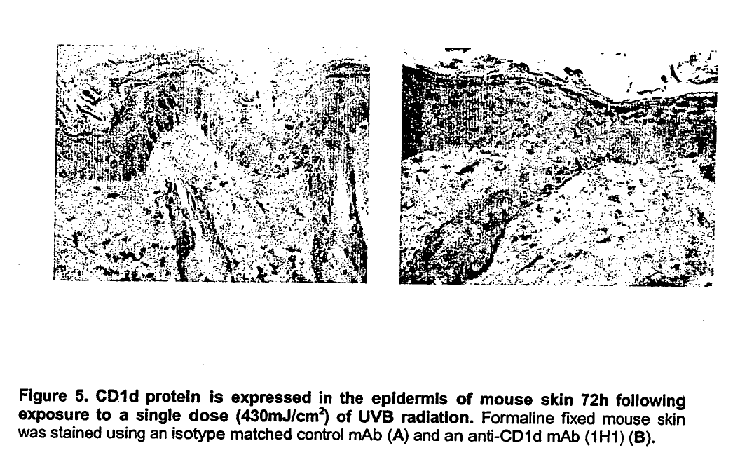

While studies of CD_{1d} proteins in the murine system suggest a widespread and constitutive expression on many hematopoietic cell types as well as intestinal epithelial cells, and hepatocytes, it was not known whether this molecule is expressed by normal and/or UV exposed mouse skin cells, especially keratinocytes. To address this question, mouse skin from unirradiated and UVB-irradiated wild-type mice was fixed in formaline, sectioned and stained using an anti-mouse CD1d mAb (1H1) to detect CD_{1d} protein. Detection of CD_{1d} protein in normal unirradiated skin was negative (data not shown). However, CD1d protein was detected (brown color) in the epidermis and dermis of UVB-irradiated mouse skin (FIG. 5). Staining was largely confined to the more differentiated layers of the skin (stratum granulosum and stratum corneum) and at the cellular level was localized to the cytoplasm and nuclear membrane.

Thus, UVB-induced mouse skin damage/burning may be directly regulated at the level of the mouse keratinocyte rather than by antigen-presenting cells (locally or systemically).

CD1d Gene Transcription is Regulated by UV Radiation

Having demonstrated that UVB-induced skin damage is regulated by CD_{1d} and that CD_{1d} protein is expressed by mouse epidermal cells (keratinocytes), it was next important to establish whether skin CD_{1d} expression is regulated by UV radiation. Any indication that skin CD_{1d} expression is regulated by UV radiation would suggest that modulation of CD_{1d} levels in skin is a critical factor responsible for regulating UV-induced skin damage. To address this question, the shaved dorsum of wild-type mice was exposed to a single dose (86 mJ/cm²) of UVB radiation and at various times post irradiation (6, 24, 48, 72 and 96 h) the irradiated skin was excised, RNA extracted and purified, and CD_{1d} mRNA levels determined by semi-quantitative RT-PCR. As a control, normal non-irradiated mouse skin was excised, RNA extracted and purified and CD1d mRNA levels determined by semi-quantitative RT-PCR.

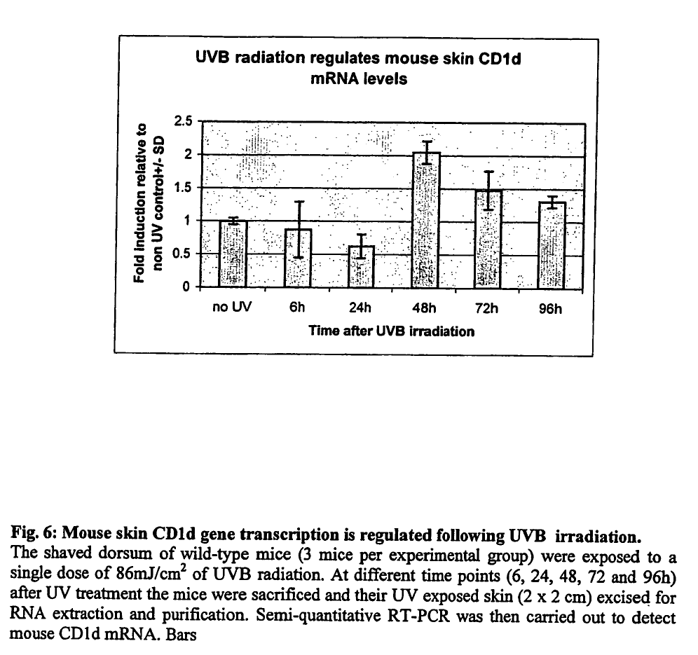

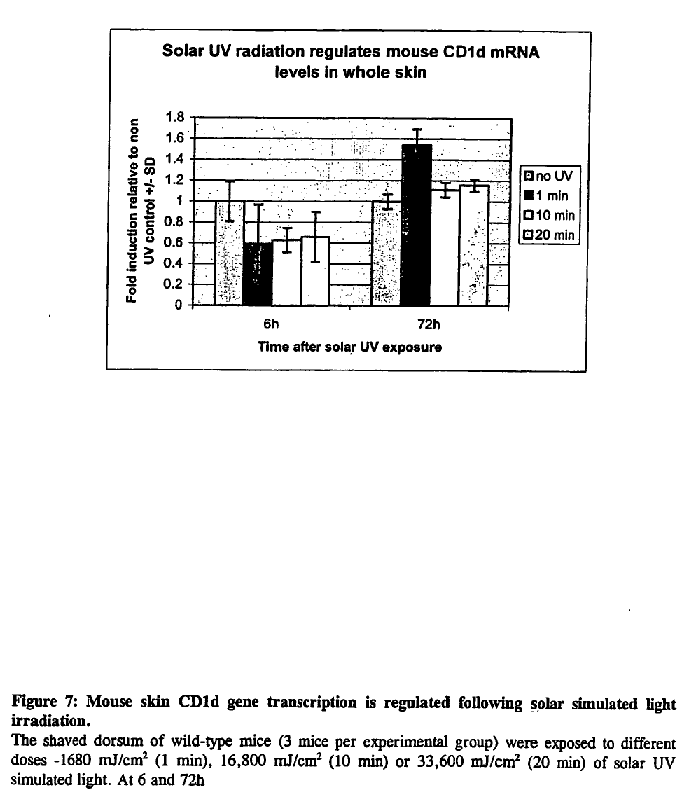

As shown in FIG. 6, the level of CD_{1d} mRNA in whole mouse skin which decreased as early as 6 h after UVB exposure was significantly reduced 24 h post irradiation compared to levels detected in normal non-irradiated skin. In contrast, 48, 72 and 96 hours following UVB exposure CD_{1d} mRNA levels were raised above the levels detected in normal unirradiated control skin. To further validate our studies on the effect of UVB radiation on skin CD_{1d} gene transcription, we exposed the shaved dorsum of wild-type mice to varying doses of solar UV irradiation (UVB+UVA)-1680 mJ/cm² (1 min), 16,800 mJ/cm² (10 min) or 33,600 mJ/cm² (20 min) of solar UV. At 6 and 72 h post irradiation the irradiated skin was excised, RNA extracted and purified, and CD_{1d} mRNA levels determined by semi-quantitative RT-PCR (FIG. 7). As with UVB exposure, we observed a similar decrease and increase in CD_{1d} mRNA levels 6 and 72 h following solar UV irradiation, respectively, regardless of the UV dose, suggesting that the response of skin CD_{1d} gene transcription to UV radiation is an important event in the skin's response to the damaging effects of UV exposure.

Human Keratinocyte CD1d Gene Transcription is Regulated by Solar UV Radiation

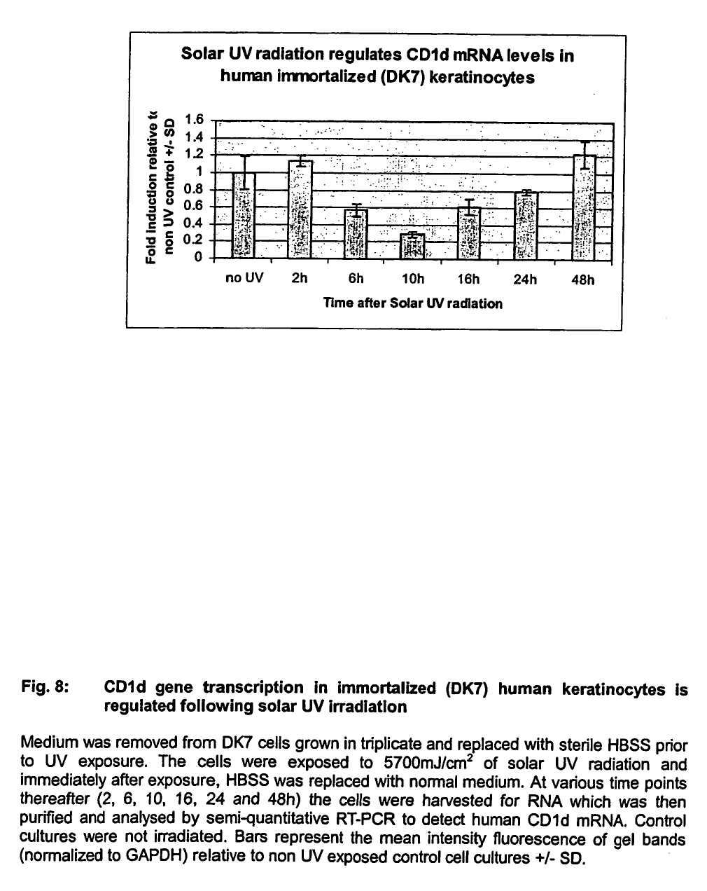

In an attempt to address whether human CD_{1d} gene transcription is regulated by UV radiation we investigated whether cultured human keratinocytes exhibit a similar gene transcription kinetic profile in response to UV irradiation. At different time points following exposure of triplicate DK7 cell keratinocyte cultures to a single dose of 5700 mJ/cm² solar UV radiation, the cells were harvested for RNA and semi-quantitative RT-PCR performed to determine the relative level of CD_{1d} mRNA (FIG. 8). As observed with UV irradiated (UVB or solar) mouse skin, CD_{1d} mRNA levels decreased 6 h post-irradiation compared to normal non-irradiated controls. Analysis of CD_{1d} mRNA levels 10 h post-irradiation revealed that these levels had further decreased compared to the levels detected in normal non-irradiated cell cultures. Between 16 and 48 h hours after UV exposure, the level of CD_{1d} mRNA increased proportionally; a pattern also observed in the skin of UV-irradiated whole wild-type skin suggesting that UV-induced CD_{1d} gene transcription in mouse skin was likely being regulated at the level of the keratinocyte.

Human keratinocyte CD_{1d} gene transcription is responsive to UV radiation and appears to be regulated in a similar manner to mouse skin CD_{1d} implying that a) skin CD_{1d} plays a critical role in regulating the response of skin to UV irradiation and b) modulation of CD_{1d} levels in skin is a critical factor responsible for regulating UV-induced skin inflammation/damage.

Gene Transcription of Key Genes which Regulate Skin Inflammation is Diminished in UVB irradiated CD1d Knockout Mouse Skin

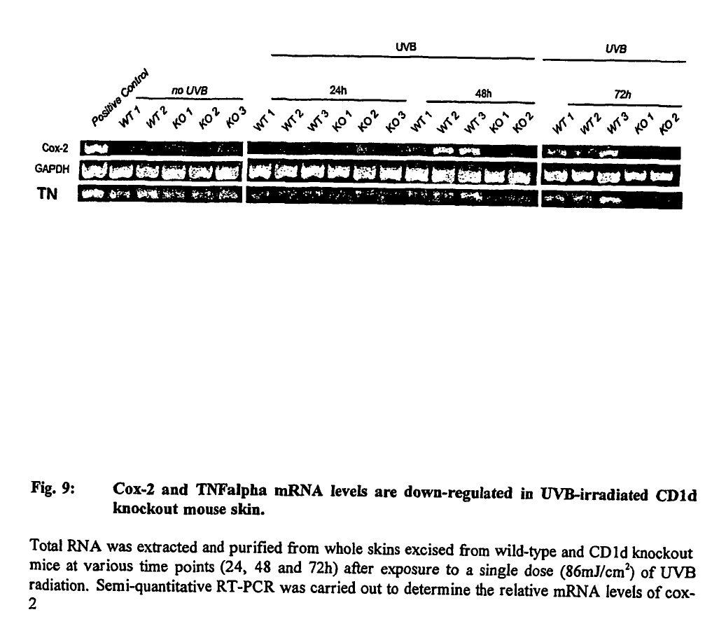

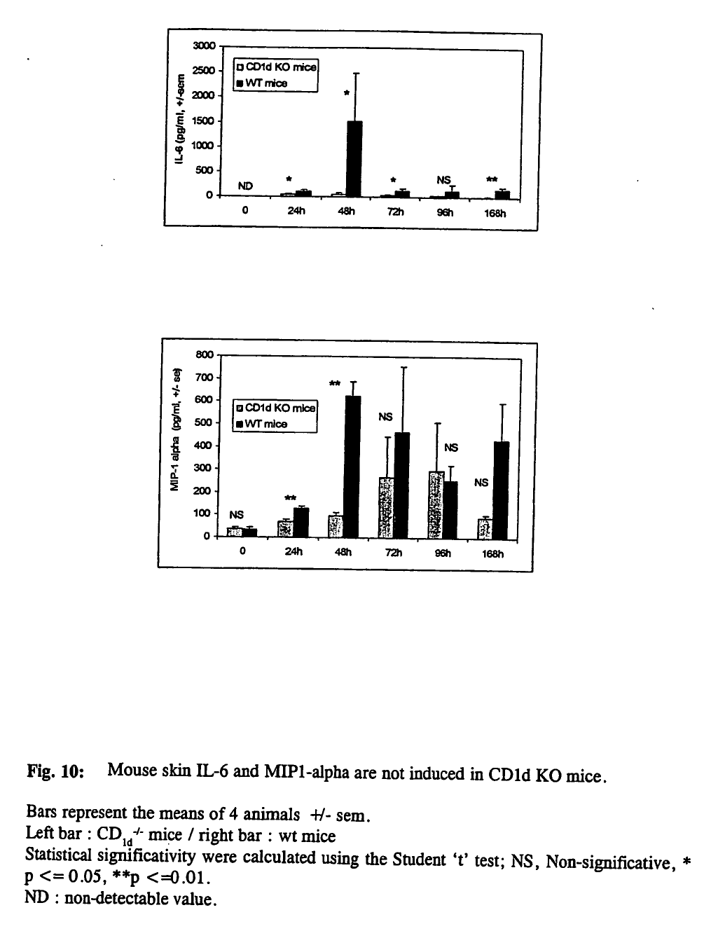

To further validate CD_{1d} as a critical molecule responsible for regulating UVB-induced skin inflammation/damage we next investigated whether COX-2 and TNF-α gene transcription (key genes responsible for regulating UVB-induced skin inflammation/damage) is deregulated in UV-irradiated CD_{1d} knockout mouse skin. It was found that COX-2 and TNF-α mRNA levels in CD_{1d} knockout mouse skin were inhibited 48 and 72 h after UV irradiation (FIG. 9). Since UV-induced skin damage/inflammation in wild-type mice is observed at 48 and 72 h after UV exposure these data demonstrate that skin CD_{1d} mediates UV-induced skin inflammation/damage by inducing COX-2 and TNF-α gene transcription.

### EXAMPLE 11

Inflammatory cytokines synthesis in UV-irradiated skin of CD_{1d} Knockout mice is decreased compared to wild-type control mice.

UV Irradiation of Mice

The mice were exposed once to an incident dose of 200 mJ/cm² UVB radiation. Three month old female inbred 129/C57BL/6 WT and 129/C57BL/6 CD_{1d} KO mice were involved in this study (n=4).

Cytokine Quantification

8 mm punch biopsies of skin were harvested at 0 h, 24 h, 48 h, 72 h, 96 h and 168 h post-irradiation and immediately frozen in liquid nitrogen. IL-6 and MIP1-alpha were quantified in skin homogenates using classical ELISA methods.

In WT mice UV-B irradiation induces a high up-regulation of inflammatory cytokines synthesis, 48 hours post-irradiation, whereas in CD_{1d} KO mice synthesis of IL-6 and MIP1-alpha protein is significantly reduced. This demonstrates a major role for CD_{1d} in UVB-induced cutaneous inflammation.

### EXAMPLE 12

Hydrocortisone down-regulates chemical stress-induced CD_{1d} gene transcription.

Primary human keratinocytes were grown in complete KGM before being exposed to 300 μM H₂O₂ as a stress factor. 48 hours after seeding, KGM medium was replaced by KGM without hydrocortisone and cultures were treated with 300 μM of H₂O₂. Total RNA was extracted at different time after treatment and CD_{1d} mRNA quantified by Real Time PCR.

For Taq Man Assays, Applied Biosystems recommends to use 10 to 100 ng of initial RNA quantity per well. Consequently, first strand cDNA synthesis was performed in a 20 μl volume using 1 μg of total RNA and 150 μg of random hexamers following the manufacturer's recommendations (Superscript™ First-Strand Synthesis System for RT-PCR, 11904-018, Invitrogen). 1 μl of the resulting cDNA samples was used for amplification by Real Time PCR.

The sets of primers and probes used for detection of CD1d cDNA were provided by Applied Biosystems as Assays on Demand (respectively Hs00174321_ml and Hs00166289_ml). The primers and probes for the housekeeping gene GAPDH were provided as PDARS (4310884E, Applied Biosystems).

All the cDNA samples were tested in triplicate. PCR reaction mixtures were prepared on ice in micro centrifuge tubes. For one replicate, a pre-mix of 24 μl was made using 1.25 μl of 20× Target or Control mix, 10.25 μl of water and 12.5 μl of 2× TaqMan Universal Master Mix, and added to 1 μl of cDNA. The PCR reaction mixtures were gently and quickly centrifuged before being aliquoted at the rate of 25 μl per well of a 96-wells plate. The plate was sealed, centrifuged at 2000 rpm for 30 seconds and placed in a 5700 Sequence Detection System for thermal cycling and fluorescence analysis using the following PCR program:

2 min at 50° C.

10 min at 95° C.

40 cycles of 15 sec at 95° C. and 1 min at 60° C.

Results were analyzed using the GeneAmp® 5700 SDS software. Amplification plots showing amplification of cDNA of interest in function of the number of cycles were obtained.

The gene expression fold changes obtained in the test condition compared to the control condition were determined by the comparative Ct method using the following formula:

$$Fold changes = {2}^{- ΔΔ Ct} = {2}^{- ( Δ Ct test - Δ Ct control )}$$

$$where$$

$$Δ Ct = {Ct}_{target gene} - {Ct}_{housekeeping gene}$$

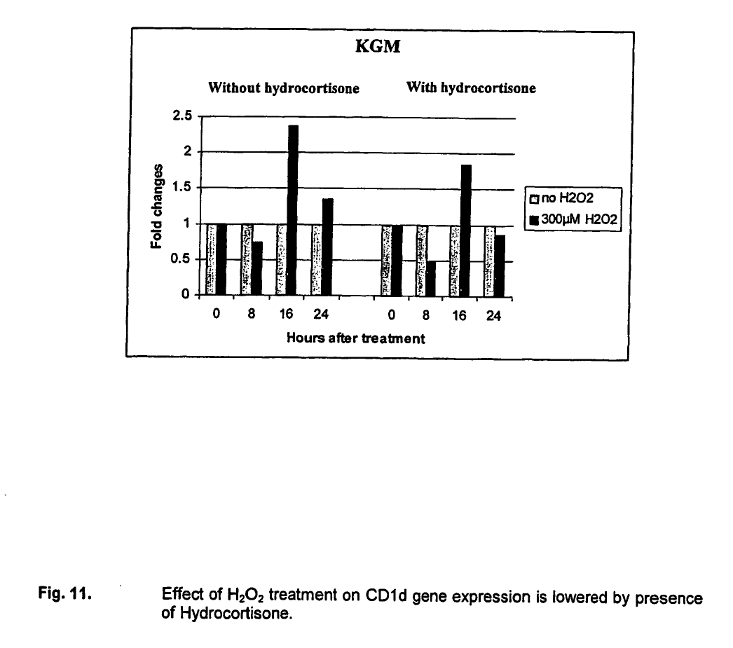

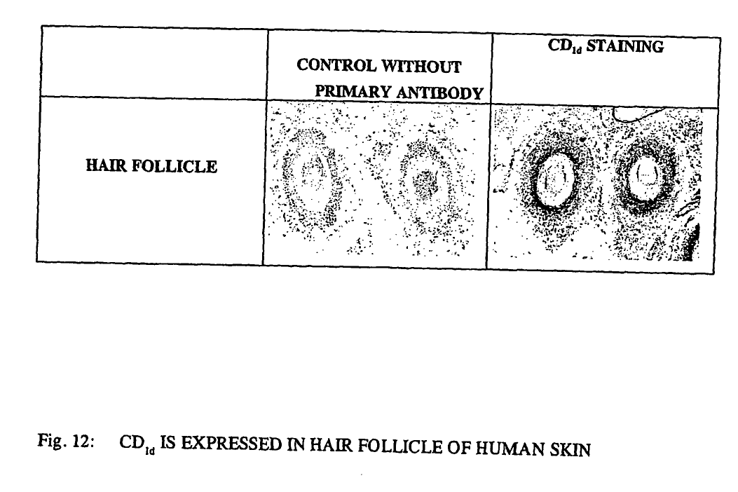

As shown in FIG. 11, modulation of CD_{1d} gene expression is observed over time when cells are exposed to H₂O₂. In presence of Hydrocortisone, the pattern of CD_{1d} gene expression obtained following H₂O₂ challenge differed in that hydrocortisone suppressed CD_{1d} transcription.

Thus, Hydrocortisone is able to down-regulate CD_{1d} expression in cells subjected to a stress.

### EXAMPLE 13

Phospholipid levels are disregulated in the skin and the intestine of CD_{1d} knockout mice compared to wild-type skin.

Mice: Female inbred C57BL/6 CD1d −/− and wild-type C57BL/6 mice aged 5 months were sacrificed and the respective tissue excised. The pieces of skin/intestinal tissue from each mouse were snap frozen using liquid nitrogen. They were then analysed for lipid content.

The main family of lipids regulated by CD_{1d} in skin were phospholipids.

nMolar per gram of skin tissue

| | CD1d−/−* | Wild-type |
|---|---|---|
| Spingomyelin | 33 +/− 30.4 | 176 +/− 116 |
| Lysophosphatidylcholine | 3.8 +/− 3.7 | 10.0 +/− 4.1 |
| Phoshatidylcholine | 35.1 +/− 21.1 | 8.5 +/− 12.9 |
| Phosphatidylserine | 67 +/− 30.4 | 115 +/− 33 |

*The values are statistically significantly different from wild-type control (p < 0.05)

Sphingomyelin

It is a ubiquitous component of animal cell membranes, where it is by far the most abundant sphingolipid. Indeed, it can comprise as much as 50% of the lipids in certain tissues, though it is usually less abundant than phosphatidylcholine. For example, it makes up about 10% of the lipids of brain. It is the single most abundant lipid in erythrocytes of most ruminant animals, where it replaces phosphatidylcholine entirely. In this instance, there is known to be a highly active phospholipase A that breaks down the glycerophospholipids, but not sphingomyelin. Like phosphatidylcholine, sphingomyelin tends to be most abundant in the plasma membrane, and especially in the outer leaflet, of cells.

Now, it is known that sphingomyelin (and other sphingolipids) and cholesterol may be located together in specific sub-domains (‘rafts’ or related structures termed ‘caveolae’) of membranes. As sphingolipids containing long, largely saturated acyl chains, they pack more tightly together, thus giving sphingolipids much higher melting temperatures than membrane glycerophospholipids. This tight acyl chain packing is essential for raft lipid organization, since the differential packing facility of sphingolipids and phospholipids is believed to lead to phase separation in the membrane, giving rise to sphingolipid-rich rafts (‘liquid-ordered’ phase) surrounded by glycerophospholipid-rich domains (‘liquid-disordered’ phase). Interactions between specific cellular proteins and lipids in these rafts are believed to be important in signalling mechanisms implicating an important role for sphingomyelin in regulating cell signaling.

Sphingomyelin is a Key Lipid in Signal Transduction Processes Involved in Apoptosis.

Also, sphingomyelin serves as a precursor for ceramides, long-chain bases and sphingosine-1-phosphate, as part of the ‘sphingomyelin cycle’, and many other important sphingolipids. (see figure below). Some of these have functions as intracellular messengers, and others are essential membrane constituents 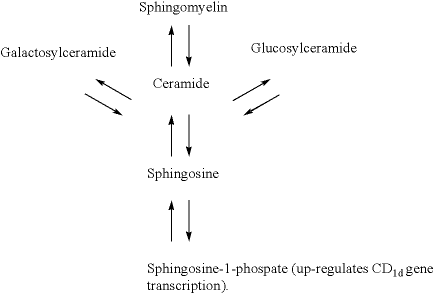
Lysophosphatidylcholine
a phospholipid that is pro-inflammatory
elevated in lesional psoriasis
intracutaneous injection induces skin inflammation
formed by the action of phospholipaseA2 which is the rate limiting step in the production of arachadonic acid. (link to regulation of COX-2 by CD₁d).

For the large intestine the following results were obtained:

| | | | Large Intestine | |
|---|---|---|---|---|
| | | | nMoles per gram of Tissue | |
| Lipid Class | Saturation | Fatty Acid Family | CD1d−/−* | Wild-type |
| Cardiolipin | Saturated | Stearic Acid | 405 ± 89 | 950 ± 442 |
| | | Total | 405 | 950 |
| | Unsaturated | Vaccenic Acid | 74 ± 21 | 167 ± 45 |
| | | Oleic Acid | 216 ± 42 | 377 ± 86 |
| | | α Linolenic Acid | 23 ± 9 | 43 ± 9 |
| | | DHA | 164 ± 92 | 596 ± 278 |
| | | Linoleic Acid | 781 ± 210 | 2066 ± 727 |
| | | DGLA | 36 ± 12 | 60 ± 16 |
| | | Total | 1294 | 3309 |
| Total Cardiolipin | | | 1699 | 4259 |
| Lysophosphatidylcholine | Saturated | Myristic Acid | 78 ± 41 | 27 ± 5 |
| | | Arachidic Acid | 7 ± 3 | 3 ± 0.5 |
| | | Total | 85 | 30 |
| | Unsaturated | Eicosenoic Acid | 9 ± 5 | 3 ± 2 |
| | | Erucic Acid | 234 ± 143 | 17 ± 36 |
| | | Eicosadienoic Acid | 153 ± 98 | 30 ± 61 |
| | | DGLA | 11 ± 5 | 4 ± 1 |
| | | Docosadienoic Acid | 12 ± 8 | 2 ± 3 |
| | | Total | 419 | 56 |
| Total Lysophosphatidylcholine | | | 504 | 86 |
| Free Fatty Acids | Saturated | Pentadecanoic acid | 31 ± 10 | 17 ± 5 |
| | | Total | 31 | 17 |
| | Unsaturated | Nervonic acid | 9 ± 5 | 2 ± 4 |
| | | Total | 9 | 2 |
| Total Free Fatty Acids | | | 40 | 19 |
| Cholesterol Ester | Unsaturated | Eicosapentaenoic acid | 5 ± 3 | 1 ± 3 |
| | | Palmitelaidic acid | 49 ± 27 | 13 ± 8 |
| | | Total | 54 | 14 |
| Diglyceride | Unsaturated | Eicosenoic acid | 11 ± 8 | 33 ± 16 |
| | | Total | 11 | 33 |
| Phosphatidylcholine | Unsaturated | Linoleic acid | 2551 ± 2321 | 6398 ± 2862 |
| | | Total | 2551 | 6398 |
| Phosphatidylserine | Unsaturated | Linoleic acid | 501 ± 105 | 1102 ± 338 |
| | | Total | 501 | 1102 |

*All values were statistically significantly different from wild-type groups (p < 0.05)

For the small intestine the following results were obtained:

| | | | Small Intestine | |
|---|---|---|---|---|
| | | Fatty Acid | nMoles per gram of Tissue | |
| Lipid Class | Saturation | Family | CD1d−/−* | Wild-type |
| Cardiolipin | Unsaturated | Eicosenoic | 12 ± 7 | 21 ± 3 |
| | | acid | | |
| | | DHA | 178 ± 124 | 480 ± 121 |
| | | Total | 190 | 501 |
| Phosphati- | Saturated | Behenic acid | 13 ± 10 | 27 ± 10 |
| dylcholine | | Total | 13 | 27 |
| | Unsaturated | Mead acid | 7 ± 4 | 15 ± 4 |
| | | Eicosate- | 2 ± 3 | 7 ± 3 |
| | | traenoic | | |
| | | acid | | |
| | | DGLA | 154 ± 96 | 310 ± 115 |
| | | Docosadie- | 5 ± 4 | 10 ± 3 |
| | | noic acid | | |
| | | Total | 168 | 342 |

| | | |
|---|---|---|
| Total Phosphatidylcholine | 181 | 369 |

*All values were statistically significantly different from wild-type groups (p < 0.05)

This above data support a role for CD_{1d} in the regulation of phospholipid metabolism which controls inflammatory processes.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A substance capable to block or modify endogenous CD_{1d} function, obtainable by a process comprising the steps of:
(a) exposing epithehal cells to a substance of interest,
(b) subjecting the epithelial cells to a stress situation,
(c) determining the effect of said stress to said epithelial cells by screening for one or more of the following assays,
(i) epithelial hyperplasia (H&E),
(ii) epithelial proliferation (BrUd, PCNA),
(iii) epithelial apoptosis (TUNEL),
(iv) p53 mutation accumulation,
(v) quantitative and qualitative assessment of epithelial lipids,
(vi) co-clustering patterns of apoptotic and non-apoptotic cell surface receptors,
(vii) production of pro-inflammatory cytokines,
(viii) production of immuno-modulatory cytokines,
(ix) markers of inflammation,
(x) anti-apoptotic transcription factors,
(xi) markers of ageing,
(d) comparing the results obtained with a control.

2. The substance according to claim 1, which is capable of preventing and/or treating detrimental effects of stress to epithelial cells.

3. The substance according to claim 1, which is capable of preventing or treating hair loss.

4. The substance according to any of the preceding claims, which is a compound reducing the transcription and/or translation of the CD_{1d} gene.

5. The substance according to claim 4, which is a polynucleotide antisense to a sequence comprised by the CD_{1d}-gene and/or the CD_{1d}-mRNA.

6. The substance according to any of the claims 1 to 4, which is a polynucleotide antisense to a sequence comprised by the glucosylceramide synthase gene and/or the glucosyl-ceramide synthase mRNA.

7. The substance according to any of the claims 1 to 4, which is a polynucleotide sense to a sequence comprised by the sphingomyelinase or ceramide synthase gene and/or the sphingomyelinase or ceramide synthase mRNA.

8. The substance according to any of the claims 1 to 4, which is a polypeptide or peptide, binding to CD_{1d} and essentially blocking or modifying CD_{1d} function.

9. The substance according to claim 8, wherein the polypeptide is an antibody or the variable part of an antibody.

10. The substance according to any of the claims 1 to 4, which is a lipid.

11. The substance according to claim 10, wherein the lipid is a sphingolipid, glycosphingolipid, phospholipid, ganglioside, sterol, fatty acid, glyceride or phosphatidylinositol phosphate.

12. The substance according to claim 10 and 11 which is derived from plants, microbes or animals, or a phytochemicals, especially a natural or synthetic polyphenols, or ingredients of green tee, and a ginkolide, vitamin, amino acid or carotenoid.

13. The substance according to claim 8, which is a ceramide or a ligand of a receptor belonging to the TNF-superfamily, in particular CD95/APO-1/Fas.

14. The substance according to any of the preceding claims for the preparation of a carrier for the prevention and/or treatment of the detrimental effects of stress to epithelial cells and/or hair loss.

15. A composition, containing at least a substance according to any of the preceding claims.

16. A composition according to claim 15, which is a food composition, a cosmetic composition or a pharmaceutical composition.

17. The composition according to claim 16, which is milk, yogurt, curd, cheese, fermented milks, milk based fermented products, ice-creams, milk based powders, infant formulae, cereal products, fermented cereal based products, mineral water, chocolate or pet food, or lotions, shampoos, creams, sun-screens, after-sun creams, anti-ageing creams and/or ointments or tablets, liquid, dried oral supplement, wet oral supplement, dry tube-feeding or wet tube-feeding or an anti-cancer drug.

18. Use of a substance according to any of the claims 1 to 14 or a composition according to any of the claims 13 to 15 for the prevention and/or treatment of damages in epithelial tissues produced by a stress situation and/or for the prevention and/or treatment of hair loss.

19. The use according to claim 18, wherein the stress, situation is a chemical stress, a biological stress or a physical stress.

20. The use according to any of the claims 19, wherein the chemical stress is exerted by exposure to oxidants or carcinogens, or wherein the biological stress is exerted by exposure to bacteria, viruses, fungi, lipids derived from surrounding cells and/or microbes, or wherein the physical stress is exerted by exposure to UV-irradiation.

21. The use according to any of the claims 18 to 20, wherein the damage is skin burning and/or blistering, cataract formation, epidermal hyperplasia, cancer, inflammation, immune suppression, skin ageing.

22. The use according to any of the claims 18 to 21, wherein the epithelial cells are derived from the skin, gut, eye, lung, prostate, liver, breast, kidney and/or the uterus.

23. The use according to claim 21, wherein the cancer is breast cancer, colon cancer, prostate cancer, liver cancer, pancreatic cancer, kidney cancer, non-melanoma and melanoma skin cancers.

24. A method for identifying CD_{1d} blocking or modifying substances, which comprises the following steps:
(a) exposing epithelial cells to a substance of interest,
(b) subjecting the epithelial cells to a stress situation,
(c) determining the effect of said stress to said epithelial cells by screening for one or more of the following assays,
(i) epithelial hyperplasia (H&E),
(ii) epithelial proliferation (BrUd, PCNA),
(iii) epithelial apoptosis (TUNEL),
(iv) p53 mutation accumulation,
(v) quantitative and qualitative assessment of epithelial lipids,
(vi) co-clustering patterns of apoptotic and non-apoptotic cell surface receptors,
(vii) production of pro-inflammatory cytokines,
(viii) production of immuno-modulatory cytokines,
(ix) markers of inflammation,
(x) anti-apoptotic transcription factors,
(xi) markers of ageing,
(d) comparing the results obtained with a control.

25. The method according to claim 24, wherein the stress situation is a chemical stress, a biological stress or a physical stress.

26. The method according to claim 25, wherein the chemical stress is exerted by exposure to oxidants or carcinogens, or wherein the biological stress is exerted by exposure to bacteria, viruses, fungi, lipids derived from surrounding cells and/or microbes, or wherein the physical stress is exerted by exposure to UV-irradiation.

27. The method according to claim 24 to 26, wherein the pro-inflammatory cytokines are selected from the group consisting of IL-1, TNF-α, PGE-2, IL-6, IFN-γ or IL-8.

28. The method according to any of the claims 24 to 26, wherein the immuno-modulatory cytokines are selected from the group consisting of PAF, IL-10, IL-4 or TGF-13.

29. The method according to any of the claims 24 to 26, wherein the lipids are selected from the group consisting of phospholipids, sphingolipids and glycosphingolipids.

30. The method according to any of the claims 24 to 26, wherein the markers of inflammation include COX-2 and iNos.

31. The method according to any of the claims 24 to 26, wherein the anti-apoptotic transcription factors include AP-1 and NFkappaB.

32. The method according to any of the claims 24 to 26, wherein the markers of aging include elastases, collagenases, metalloproteinases, gelatinases, stromelysins, telomerases.

33. Use of a substance according to any of the claims 1 to 14 or a composition according to any of the claims 15 to 17 for decreasing multi-drug resistance of cancers.

34. The use according to claim 33, wherein the cancer is skin, gut or breast cancer.

35. Use of cells expressing and/or over-expressing CD_{1d} in an assay for screening for substances modifying and/or blocking CD_{1d} function.

36. Use of CD_{1d}^{-/-} animals as a test model for determining the activity of substances influencing damages in epithelial tissues produced by a stress situation and/or hair loss.

37. Use of a substance according to any of the claims 1 to 14 in gene therapy.
